(19) United States
(12) Patent Application Publication
(10) Pub. No.: US 2025/0325269 A1
(43) Pub. Date: Oct. 23, 2025

(54) OCCLUSION BALLOONS FOR MODULATING BLOOD FLOW

(71) Applicant: EDWARDS LIFESCIENCES CORPORATION, Irvine, CA (US)

(72) Inventors: Aviran Pitusi, Rosh Haayin (IL); Amir Davidesko, Binyamina Givat Ada (IL); Avraham Zakay (aka Zakai), Tel Aviv-Yafo (IL); John Richard Carpenter, Irvine, CA (US)

(73) Assignee: EDWARDS LIFESCIENCES CORPORATION, Irvine, CA (US)

(21) Appl. No.: 19/182,529

(22) Filed: Apr. 17, 2025

Related U.S. Application Data
(60) Provisional application No. 63/637,303, filed on Apr. 22, 2024.

(51) Int. Cl. A61B 17/12; A61B 90/00

(52) U.S. Cl. CPC A61B 17/12109; A61B 17/12036; A61B 17/12136; A61B 2090/064

(57) ABSTRACT

Implantable devices are described for modulating blood flow in a blood vessel. The devices may include an expandable member and an inflation line. The devices may further include an anchor for securing the inflation line along an inner wall of the blood vessel. The expandable member is adjustable for modulating blood flow through the blood vessel. Such a device may be useful for reducing peak blood pressures in the heart, thereby reducing symptoms associated with congestive heart failure.

### CROSS-REFERENCE TO RELATED APPLICATIONS

This application claims the benefit of U.S. Provisional Patent Application No. 63/637,303 filed on Apr. 22, 2024, the contents of which are hereby incorporated by reference.

### BACKGROUND

This disclosure relates generally to the field of medical devices and procedures, and more specifically to the field of blood flow management in blood vessels.

Conventional medical devices that utilize balloon-based occlusion are typically manufactured to inflate a balloon that remains in a static location within a blood vessel for a short duration during an implantation procedure. The balloon of such conventional devices is then removed after implantation and/or treatment during the procedure

### SUMMARY

The techniques described herein relate to implantable devices for modulating blood flow in a blood vessel. One exemplary device includes an expandable member having an inflation port coupled to an inflation line, and an anchor (e.g., anchor frame, stent, etc.) for securing the inflation line along an inner wall of the blood vessel. The expandable member is provided to modulate blood flow through the blood vessel. Such a device may be useful for reducing peak blood pressures in the heart, thereby reducing symptoms associated with congestive heart failure.

In some aspects, the techniques described herein relate to an implantable flow restrictor for a blood vessel, the implantable flow restrictor including an outer frame, an expandable member including an inflation port coupled to a first portion of an inflation line, the expandable member defining a volume configured to receive an inflation fluid therein through the inflation port, and an anchor configured to couple a second portion of the inflation line to an internal surface of the outer frame to allow the expandable member to move (e.g., oscillate) within the outer frame or within a portion of the blood vessel in response to a blood flow (e.g., laminar and/or turbulent blood flow) through the blood vessel.

In some aspects, the techniques described herein relate to an implantable device for modulating blood flow in a blood vessel, the device including an outer frame defining an internal surface, an expandable annulus member at least partially coupled to the internal surface of the outer frame, the expandable annulus member fluidly coupled to a first inflation line, the expandable annulus member defining an annulus volume configured to receive an inflation fluid therein through the first inflation line, wherein the expandable annulus member defines a device orifice through which fluid flows.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates a perspective view of an example device for modulating blood flow through a blood vessel.

FIG. 1B illustrates a top-down perspective view of an example device for modulating blood flow through a blood vessel.

FIG. 2A illustrates an embodiment of a device for modulating blood flow implanted in a blood vessel and electrically coupled to a control device.

FIG. 2B illustrates an embodiment of a device for modulating blood flow implanted in a blood vessel and electrically coupled to a control device.

FIG. 3A illustrates a bottom-up perspective view of an example flow modulation device in a deflated state.

FIG. 3B illustrates a schematic representation of an anchored flow modulation device in a deflated state.

FIG. 3C illustrates a bottom-up perspective view of the device shown in FIG. 3A in an example partially expanded configuration.

FIG. 3D illustrates a schematic representation of an anchored flow modulation device in an expanded configuration.

FIG. 3E illustrates a top-down perspective view of the device shown in FIG. 3A in a deflated state.

FIG. 3F illustrates a top-down perspective view of the device shown in FIG. 3A in an example partially expanded configuration.

FIG. 3G illustrates a top-down perspective view of the device shown in FIG. 3A in another example partially expanded configuration.

FIG. 3H illustrates a top-down perspective view of the device shown in FIG. 3A in a fully expanded configuration.

FIG. 4A illustrates another schematic representation of an anchored flow modulation device in a deflated state.

FIG. 4B illustrates another schematic representation of an anchored flow modulation device in an expanded configuration.

FIGS. 5A-5B illustrate a schematic representation of the example device of FIGS. 4A-4B within a blood vessel.

FIGS. 6A-6B illustrate a schematic representation of the example device of FIGS. 4A-4B including an inner surface coating.

FIGS. 7A-7C illustrate example expandable members for use with any of the devices described herein.

FIG. 8 illustrates a block diagram of an example system for modulating blood flow through one or more blood vessels.

FIG. 9 illustrates a schematic diagram of an example embodiment of a system for modulating blood flow through a blood vessel.

FIG. 10 illustrates a flow diagram of an example process of modulating blood flow through one or more blood vessels.

FIG. 11 illustrates a schematic representation of portions of a human subject in which the devices described herein may be implanted.

FIG. 12 illustrates a perspective view of an example flow modulation device.

FIG. 13 illustrates a cross-sectional view of the flow modulating device of FIG. 12.

FIG. 14 illustrates a top perspective view of the flow modulating device of FIG. 12.

FIG. 15 illustrates a cross-sectional view of an embodiment of a flow modulating device.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure and protection to these embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the devices and methods described herein may enable modulating and/or balancing of blood flow through a blood vessel using expandable members such as balloons, rings, flexible devices having one or more enclosures, inflatable devices having one or more enclosures, or other expandable and/or formable devices that may take more than one form. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body. In some examples, the devices described herein may function to limit flow through a Superior Vena Cava (SVC) when right atrial pressure exceeds a predefined threshold pressure in order to decrease total blood flow into the right atrium of the heart and thus reduce renal venous pressure. In some examples, the devices described herein may be flow modulation devices that include at least one expandable member for selectively restricting blood flow from the SVC to the right atrium of the heart. Such expandable members may include one or more expandable balloons or balloon-like devices.

The examples presented herein may relate to providing devices, methods, and/or methods of treatment (MOTs) for modulating, regulating and/or otherwise managing blood flow to or through one or more blood vessels. The terminology of restricting blood flow, regulating blood flow, modulating blood flow, managing blood flow, and balancing blood flow causes regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as a vena cava, the SVC and an Inferior Vena Cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or congestive heart failure (CHF). For example, patients with CKD and/or CHF may exhibit reduced kidney function when pressure in the right atrium of the heart is above a predefined pressure threshold. The predefined pressure threshold may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrial pressure increases and/or pressure variations. In particular, the devices, methods, and/or MOTs described herein can be used to reduce and maintain low pressure in the right atrium, which provides a technical effect of enabling the kidneys to more effectively filter blood.

The devices, methods, and/or MOTs described herein may utilize inflatable devices implanted in a blood vessel and functionable to perform blood flow management actively and/or passively to assist in reducing and/or maintaining right atrium pressures and/or renal pressures at a relatively low pressure even when a surge in blood volume occurs in one or more vessels of the venous system. In some embodiments, the devices described herein are temporarily implanted to perform blood flow management actively and/or passively to assist in reducing and/or maintaining right atrium pressures and/or renal pressures and may be removed at some point after completion of blood flow management treatment(s).

In general, managing blood flow through a blood vessel can be achieved by the devices described herein by providing a plurality of flow modulation states (e.g., device configurations). For example, the flow modulating devices described herein can include one or more expandable members or expandable annulus members that can each be inflated to one of a plurality of inflation states to modulate flow through the flow modulating device and therefore in a vessel within which the device is installed or in a vessel in fluid communication with the vessel in which the device is installed. The amount of flow and/or pressure in a vessel can be highly tuned or modulated based on the inflation state of any or all expandable members or expandable annulus members in the devices described herein. An inflation state of an expandable member or an expandable annulus member may be based on a blood pressure in a vessel or another parameter of the vessel. A predetermined inflation state of an expandable member or an expandable annulus member may be based on a blood pressure in the vessel, such that the pressure in the expandable member or the expandable annulus member in the predetermined inflation state exceeds the blood pressure in the vessel. Additionally, or alternatively, a predetermined inflation state of an expandable member or the expandable annulus member may be based on an inflation volume of the expandable member or the expandable annulus member and/or a desired cross-sectional area reduction (or a desired cross-sectional area increase) of a cross-section of the lumen of a frame to which the expandable member or the expandable annulus member is coupled.

### Systems and Devices

Disclosed herein are systems and methods for modulating blood flow through a blood vessel. In some examples, the systems include implantable flow modulating devices used in blood flow occlusion therapy. For example, the devices described herein may relate to venous occlusion therapy using implantable and/or electronically controlled flow restricting devices for the treatment of acute heart failure. Some devices may be non-implantable or partially implantable. In some examples, the devices described herein generally function to occlude or partially occlude a blood vessel, such as the SVC, the IVC, and/or other vessels or junctions (e.g., a vena cava, an azygos junction, etc.). In such examples, blocking or occluding such junctions or other vessels can ensure that blood may not bypass the devices described herein, but instead flow through an inner diameter of such devices. In some examples, the devices described herein have been contemplated for use in a subject (e.g., patient) having chronic heart failure and/or chronic kidney disease but may be used in any vessel for flow regulation therethrough. Variations in occlusion (e.g., changes in expandable member size) may occur over longer times (e.g., hours or days) or much shorter times (e.g., minutes or even seconds, such as where cyclical changes occur responsive to heart beat). Longer term modulation of blood flow may help bring blood pressures back into equilibrium and thereby reduce symptoms of heart failure.

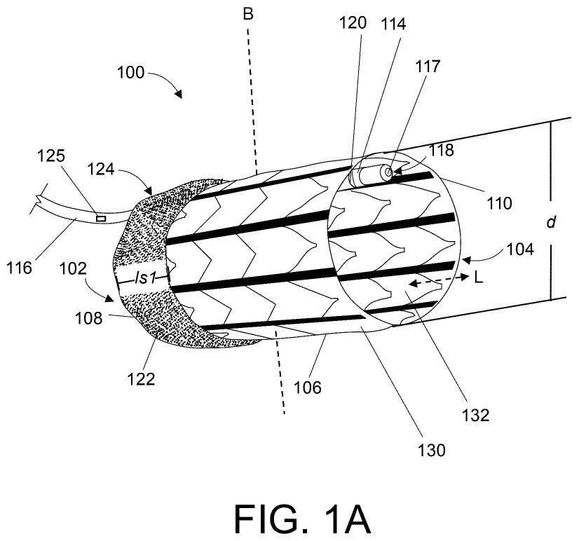

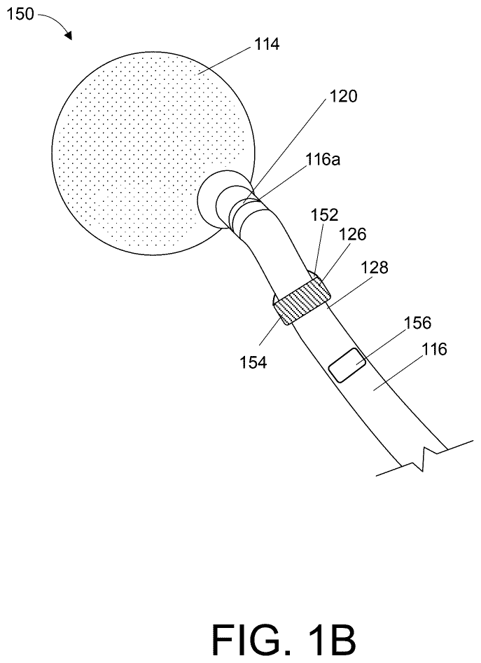

FIGS. 1A-1B illustrate views of an example flow modulating device 100 for modulating blood flow through a blood vessel. The device 100 may be implanted into a blood vessel, such as the SVC, the IVC, or any other blood vessel where modulating blood flow is desired. For example, because blood flows into the right atrium through both the SVC and the IVC, reduction of renal venous pressure can improve GFR and reduce venous congestion. The device 100 may reduce such venous congestion by limiting flow through the SVC when right atrial pressure is high, thereby decreasing total flow into the right atrium, which may reduce renal venous pressure. For example, the device 100 may modulate a volume of blood flowing from the SVC into a right atrium using one or more expandable members of device 100 to decrease right atrial pressure and/or to cause a decrease in renal pressure. In general, one or more expandable members of the device 100 may be reduced in size (e.g., deflated, collapsed, etc.) to reestablish flow of blood through the blood vessel after decreasing right atrial pressure and/or renal pressure.

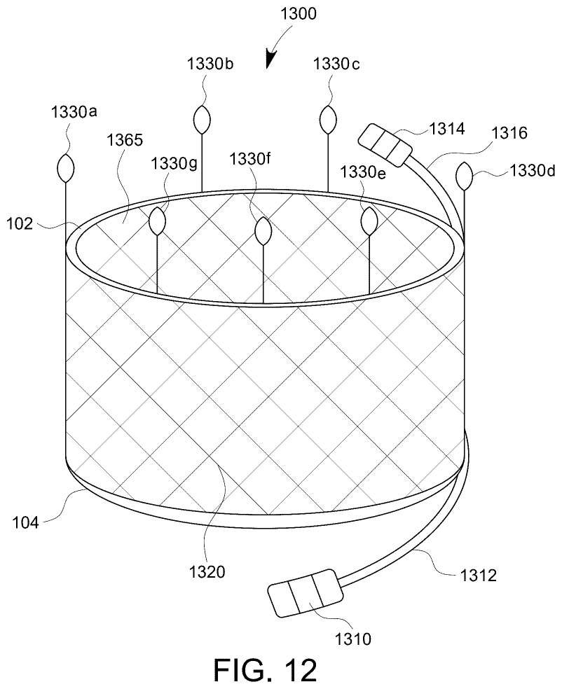

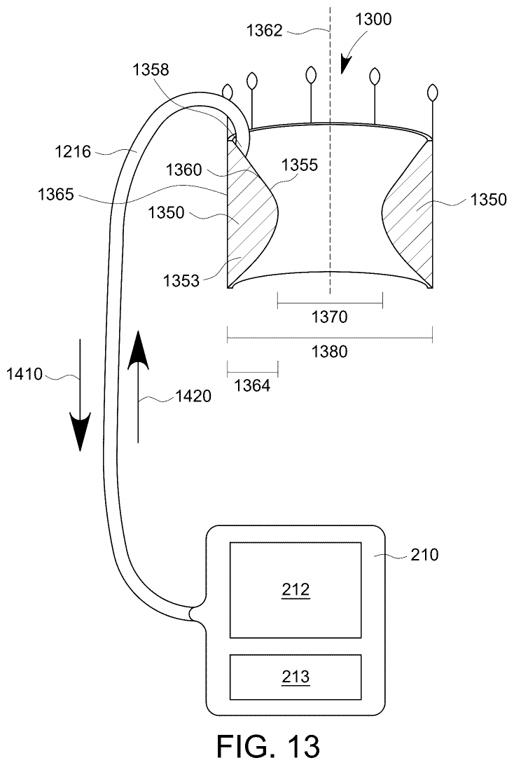

In some examples, the device 100 may include a self-expanding outer frame (e.g., frame, stent, braid, etc.) that may be delivered into the blood vessel (e.g., via jugular access, subclavian access, or transfemoral access) using a sheathed catheter (not shown). In some examples, the outer frame may be partially or fully encased in at least one polymeric layer. In some examples, the device 100 includes one or more controls for expanding and constricting (uniformly or nonuniformly) a perimeter of an end portion of the outer frame. In some examples, the device 100 further includes a skirt membrane wrapped around at least a portion of an exterior surface of the outer frame. In some examples, the device 100 does not include an outer frame and is instead delivered into the blood vessel as an anchor and one or more expandable members. In some examples, the device 100 further includes one or more recapture features, as described for example with respect to FIGS. 12-13. In some examples, the one or more expandable members of device 100 may be one or more expandable annulus members. The one or more expandable annulus members may include one or more inflatable inner cavities, as described with respect to FIGS. 13-15.

FIG. 1A illustrates a perspective view of an example device 100 for modulating blood flow through a blood vessel. The blood vessel may be a vena cava, the SVC, or the IVC, or adjacent vessel. The device 100 includes an outer frame 106 that may surround or at least partially surround one or more expandable members 114 (shown partially expanded in FIG. 1B). The outer frame 106 may be expandable and/or contractable along all or a portion of the frame. The outer frame 106 includes an inflow end 102 and an outflow end 104, with a lumen and a longitudinal axis (L) extending therethrough. The inflow end 102 may correspond to a proximal end 108 of the device 100. Similarly, the outflow end 104 may correspond to a distal end 110 of the device 100. In general, the inflow end 102 is defined to be upstream from the outflow end 104.

In some examples, the outer frame 106 may be substantially tubular shaped with a substantially annular cross section at the inflow end 102 and the outflow end 104. In some examples, the lumen is substantially the same cross section through the outer frame 106. In some examples, the lumen has a variable cross section through the outer frame 106. For example, the lumen may be radially narrowed along an intermediate portion located between the inflow end 102 and the outflow end 104. The intermediate portion may be configured to constrain the expandable member 114, for example, because the intermediate portion may have an annular cross section that is less than an annular cross section at the inflow end 102 (e.g., proximal end 108) or the outflow end 104 (e.g., distal end 110).

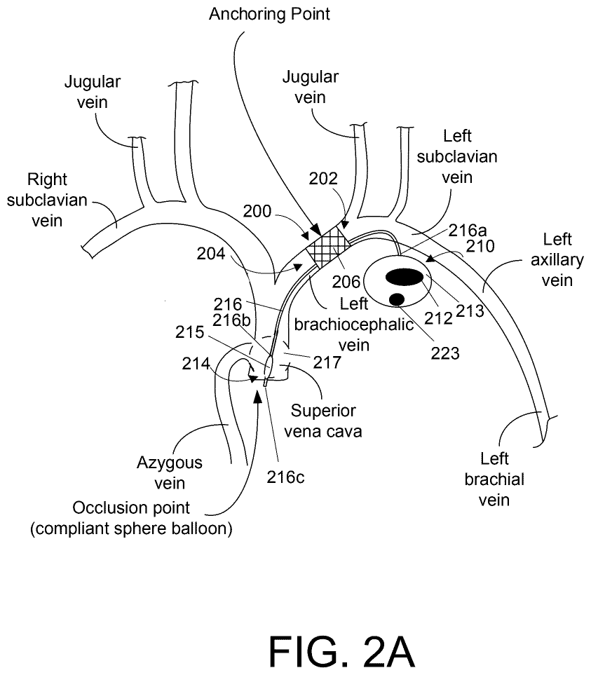

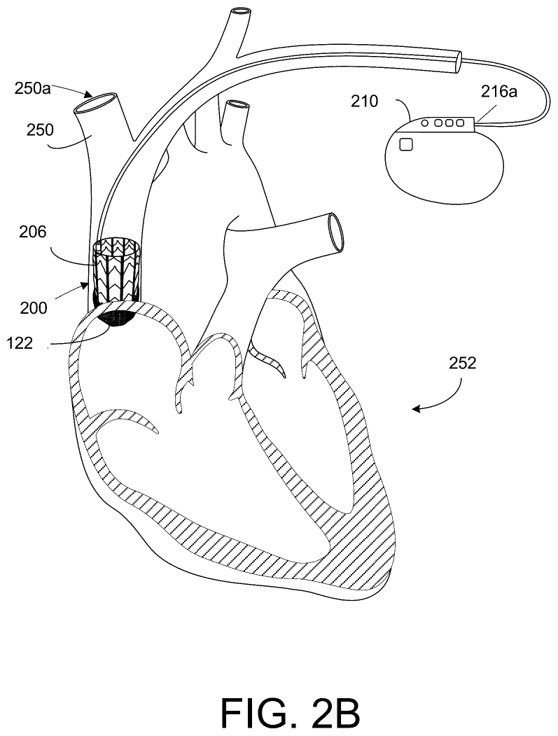

In some examples, a perimeter of the outflow end 104 may contain or at least partially contain an expandable member 114 extending from an inflation line 116. The inflation line 116 may be flexible. The inflation line 116 may be a tube or other shape that may include a lumen to allow flow of air and/or fluid to flow therethrough. The inflation line 116 may extend from an actuation unit (e.g., controls 802, pump 213) that is outside the body (or alternatively, subcutaneous implanted in the body) through a lumen of the outer frame 106 and within a threshold distance of a perimeter of the distal end 110 of the frame 106. The inflation line 116 may include one or more sensors or sensor modules 117 at a distal end (e.g., distal end 216c of FIG. 2A) of the line 116 opposite a proximal end (e.g., proximal end between frame 206 and expandable member 217 in FIG. 2A) of the inflation line 116. Inflation line 116 terminates in at least one actuation unit (e.g., controls 802) which may function to trigger device 200, for example, to actuate to inflate or deflate expandable member 214.

In some examples, the inflation line 116 may be arranged to secure one or more expandable members 114 within a threshold distance of the distal end 110 of the frame 106. For example, the expandable member 114 may include an inflation port 120 coupled to a first portion 116a (FIG. 1B) of the inflation line 116. The expandable member 114 may define a volume configured to receive an inflation fluid therein. For example, the expandable member 114 may receive (or expel) an inflation fluid through the inflation port 120 to partially block or block a portion of the lumen of frame 106 (e.g., the outflow end 104 of the device 100). In some embodiments, the expandable member 114 may receive (or expel) an inflation fluid through the inflation port 120 to partially block or block (or occlude) a blood vessel in which one or more expandable members 114 is implanted.

In some variations, the one or more expandable members 114 can include an expandable balloon. In some examples, the one or more expandable members 114 can include a compliant material. In some examples, the one or more expandable members 114 can be formed of a compliant material. In some examples, the one or more expandable members 114 can consist essentially of a compliant material. A compliant material may exhibit a burst pressure of about 0 atmospheres (atm) to about 2 atm. In some examples, a compliant material may be able to expand about 20 percent to about 900 percent. Non-limiting examples of compliant materials include silicones, latex, polyvinyl chloride, polyolefin copolymer, or a combination thereof. The one or more expandable members 114 may be formed of a compliant material to provide an advantage of maintaining a smooth surface in all configurations of expansion. Such a compliant material that maintains a smooth surface during inflation and deflation may reduce or prohibit deformations in the material in which blood might be trapped and/or stagnate.

In some instances, the one or more expandable members 114 can include a semi-compliant material. In some examples, the one or more expandable members 114 can be formed of a semi-compliant material. In some examples, the one or more expandable members 114 can consist essentially of a semi-compliant material. A semi-compliant material may exhibit a burst pressure of about 1 atm to about 25.5 atm. In some examples, a semi-compliant material may be able to expand about 10 percent to about 20 percent. Non-limiting examples of semi-compliant materials include polyethylene terephthalate, nylons, thermoplastic polyurethanes, thermoplastic elastomers, or a combination thereof.

In some instances, the one or more expandable members 114 can include a non-compliant material. In some examples, the one or more expandable members 114 can be formed of a non-compliant material. In some examples, the one or more expandable members 114 can consist essentially of a non-compliant material. A non-compliant material may exhibit a burst pressure of about 1 atm to about 25.5 atm. In some examples, a non-compliant material may be able to expand about 0 percent to about 10 percent. Non-limiting examples of non-compliant materials include polyethylene terephthalate and like materials.

In some examples, one or more of the expandable members 114 include a compliant material, and one or more of the expandable members 114 include a non-compliant material. Expandable members having different properties or including different materials may, for example, achieve different filling rates of the expandable members, make a subset of the one or more expandable members more resistant to bursting, achieve various degrees of expansion of the one or more expandable members, allow differential filling of a blood vessel or a cross-sectional area of a lumen of the frame 106 based on a material of the one or more expandable materials, and the like. In some examples, one or more of the expandable members 114 may include a blend of a compliant material and a semi-compliant material. In some examples, one or more of the expandable members 114 may include a blend of a non-compliant material and a semi-compliant material. In some examples, one or more of the expandable members 114 may include a blend of a compliant material and a non-compliant material.

In some examples, the expandable member 114 may be substantially tethered or anchored to a portion of a blood vessel and/or a portion of the outer frame 106 through an intermediary member, such as the inflation line 116. For example, an anchor (e.g., anchor 126 of FIG. 1B) may be configured to couple at least a portion of the inflation line 116 to an internal surface of the outer frame 106 (or to a portion of a blood vessel or implanted device within the vessel) to allow the expandable member 114 to move (e.g., oscillate) within the outer frame 106 or within a portion of the blood vessel in response to blood flow through the blood vessel. Tethering the expandable member 114 through an intermediary member (such as the inflation line 116) can provide an advantage of anchoring part of the moveable device at a location other than on the occlusion device component performing the work (i.e., the expandable device), leaving the occlusion device component(s) to function unencumbered within the space of the vessel according to detected pressures. In addition, using an intermediary member to tether the expandable member 114 to the frame 106 can allow for the expandable member 114 to be expanded in a predictable and equilateral manner, rather than having one side or end tethered to a frame wall that may interfere with the expansion shape that the device may be designed to take. Direct tethering of the expandable member 114 to the frame 106 without an intermediary member may also cause uneven inflation of the member 114, which can result in having one side of the member 114 expanding toward a frame wall opposite the tether position rather than evenly expanding on all sides of the expandable member 114.

In some examples, the outer frame 106 includes a skirt membrane 122, which with deployment may help to create a seal between a blood vessel wall and the device 100 to prevent blood flow between the blood vessel wall and the exterior of the device 100. The skirt membrane 122 may be wrapped around an exterior surface of the coated or uncoated frame 106. The skirt membrane 122 may be disposed offset from a lateral centerline (e.g., bisect B) at a midpoint of the frame 106 and toward the inflow end 102 of the frame 106. An inflow perimeter 124 of the skirt membrane 122 may end within a threshold distance of the inflow end 102 of the frame 106. For example, the threshold distance may be about 8 mm to about 12 mm from the inflow perimeter 124; about 8 mm to about 9 mm from the inflow perimeter 124; about 9 mm to about 10 mm from the inflow perimeter 124; about 10 mm to about 11 mm from the inflow perimeter 124; or about 11 mm to about 12 mm from the inflow perimeter 124. A length ls1 of the skirt membrane 122 may be about 15 mm to about 19 mm; about 15 to about 16 mm; about 16 mm to about 17 mm; about 17 mm to about 18 mm; or about 18 mm to about 19 mm.

The skirt membrane 122 may function to reduce blood stasis and/or pooling around the implanted device 100. For example, the skirt membrane 122 may be at least partially incorporated into an inner wall of a blood vessel to reduce or eliminate clotting and/or blood stasis within cells and struts of the device 100 by providing an effective seal between the skirt membrane 122 and the blood vessel wall.

In a non-limiting example, the skirt membrane 122 may be incorporated into an inner wall of the vena cava and may be positioned to seal an entrance to at least one additional blood vessel (or junction) branching from the vena cava when the device 100 is implanted in the vena cava. For example, the skirt membrane 122 may be positioned in the IVC or the SVC and aligned to seal or block an entrance to the azygos junction. Sealing, blocking, or occluding the azygos junction can ensure that blood does not bypass the device 100, but instead flows through an inner diameter of device 100.

In some examples, the skirt membrane 122 may comprise or be formed of poly-delta-valerolactone (PVL). In some examples, the skirt membrane 122 may comprise or be formed of PVL and another polymer. In some examples, the skirt membrane 122 may comprise or be formed of mesh or braided metal that may be coated. In some examples, an inner surface of the skirt membrane 122 may be coupled to an outer polymeric layer of outer frame 106. In some examples, the skirt membrane 122 may be sewn, sutured, or otherwise affixed to a portion of an outer polymeric layer of outer frame 106.

The outer frame 106, 1320 (shown in FIG. 12) may be a stent (or braid) constructed of metal wire (e.g., stainless steel, platinum, Nitinol® wire or another shape memory alloy), or other material suitable for implantation in the human body. In some examples, the frame 106, 1320 is a bare metal stent, such that the frame 106, 1320 may be arranged to be at least partially incorporated into an inner wall of the blood vessel. In some examples, the frame 106 includes one or more additional coverings such as sleeves, skirts, coatings, linings, etc., as described elsewhere herein.

In general, the frame 106, 1320 is formed from a plurality of struts that form cells spaced from the inflow end 102 to the outflow end 104 and extending from each other around the frame 106, 1320. For example, the struts may form two or more rows of cells. In some examples, the struts may form one or more rings of cells. The rings may be stacked from the inflow end 102 to the outflow end 104. For example, a number of adjacent rows of cells formed of struts (e.g., diamond-shaped cells, polygon-shaped cells, etc.) may form the rings. For example, the frame 106 may include one or more rings of cells stacked from the inflow end 102 to the outflow end 104.

The outer frame 106, 1320 may have a length from the outflow end 104 to an end of a deployment member (e.g., expandable member 114) of about 45 mm to about 75 mm; about 45 mm to about 50 mm; about 50 mm to about 55 mm; about 55 mm to about 60 mm; about 60 mm to about 65 mm; about 65 mm to about 70 mm; or about 70 mm to about 75 mm.

The outer frame 106 may have a diameter d of about 20 mm to about 26 mm; about 21 mm to about 22 mm; about 22 mm to about 23 mm; about 23 mm to about 24 mm; about 24 mm to about 25 mm; or about 25 mm to about 26 mm.

The outer frame 106, 1320 may be covered by one or more layers of polymer (e.g., outer polymer layer 130, inner polymer layer 132) that may further extend a length of the frame 106 on the inflow end 102 and/or the outflow end 104 by an additional length of about 0.1 mm to about 5 mm; about 0.1 mm to about 0.3 mm; about 0.3 mm to about 0.5 mm; about 0.5 mm to about 1 mm; about 1 mm to about 1.5 mm; about 1.5 mm to about 3 mm; about 3 mm to about 4 mm; or about 4 mm to about 5 mm.

In examples in which the outer frame 106, 1320 is covered by one or more layers of polymer, an inner diameter and an outer diameter may vary from diameter d from 0.05 mm to about 0.75 mm.

In some examples, the outer frame 106, 1320 is substantially formed of shape memory alloy and has an inner surface substantially covered with the inner polymer layer 132 and an outer surface substantially covered with the outer polymer layer 130. The one or more polymer layers 130, 132 may represent example polymeric coverings composed of a material capable of heat shrinking and/or lamination such that portions of each covering are coupled at locations along the stent walls of frame 106, 1320. For example, the inner polymer layer 130 and the outer polymer layer 132 may comprise or be formed of thermoplastic polyurethane or polyolefin or other polymers described herein. The inner polymer layer 130 may be coupled to portions of the outer polymer layer 132 along the walls/body of frame 106, 1320. In some examples, coupling the inner polymer layer 130 to the portions of the outer polymer layer 132 may include laminating the portions of the outer polymer layer 132 to the portions of the inner polymer layer 130. In some examples, the inner polymer layer 130 is optional. In some examples, the outer polymer layer 132 is optional. For example, the inner polymer layer 130 may be coupled to portions of the skirt membrane 122 rather than portions of the outer polymer layer 132.

In some examples, the layers 130, 132 extend beyond the outflow end 104. When the layers 130, 132 are laminated (e.g., heat shrunk, adhered together, or otherwise coupled), device 100 may be arranged in a substantially tubular-shaped device with a substantially annular cross section at the inflow end 102 and outflow end 104. However, because laminations can cause shrinkage, portions of the ends 102, 104 may not be substantially annular, but may instead take the form of a substantially flower-shaped, star-shaped, or polygon-shaped cross section at the outflow end 104 and/or inflow end 102.

In some examples, the frame 106, 1320, the one or more expandable members 114 or expandable annulus members 1350 and/or one or more layers of material can include an embedded radiopaque marker. The use of the embedded radiopaque marker may increase visibility of the device 100, 1300 (or portions of device 100, 1300) using fluoroscopy during device placement in a vessel, repositioning the device in a vessel, extracting a device from a vessel, and/or routine maintenance or check-ups on the device and/or the subject. In some examples, a fluid for filling the expandable member 114 or expandable annulus member 1350 may include up to about 20 percent contrast fluid; up to about 15 percent contrast fluid; or up to about 10 percent contrast fluid such that the member 114, 1350 may fluoresce under fluoroscopy.

In general, the struts of outer frame 106, 1320 may have a variable radial stiffness along the circumference of the device 100, 1300 from the proximal end 108 to the distal end 110. For example, struts near ends 108. 110 may be of a particular width to provide a first radial stiffness against the vessel. The struts on other portions of the frame 106 moving toward bisect B, for example, may have a particular width to provide a second radial stiffness. In general, the first radial stiffness may be less than the second radial stiffness to allow for improved bending of struts near ends 108, 110.

FIG. 1B illustrates a top-down perspective view of an example device 150 for modulating blood flow through a blood vessel. The blood vessel may be a vena cava, the SVC, the IVC, or an adjacent vessel. The device 150 may include any or all of the features of device 100. In some examples, the device 150 does not include an outer frame 106 and instead is affixable to a portion of a blood vessel or another implanted device.

The device 150 may include one or more expandable members 114 having an inflation port 120 coupled to an inflation line 116 and an anchor having a first surface 152 that may be coupled to the inflation line 116 along a portion of length of the inflation line 116 and a second surface 154 that may be coupled to a wall of the blood vessel. The one or more expandable members 114 may include an inner cavity in fluid communication with the inflation line 116.

As shown, the device 150 includes the partially expanded expandable member 114 coupled to inflation port 120, which couples to inflation line 116 at or near distal end (e.g., distal end 216b in FIG. 2B) of the inflation line 116. In general, the expandable member 114 of device 100, 150 may be adjustable to any number of positions between expanded and collapsed when implanted in a blood vessel. Such positions may include at least an expanded configuration which may represent the expandable member 114 being inflated to fully occlude the blood vessel, a partially expanded configuration which may represent the expandable member 114 being inflated or deflated to partially occlude the blood vessel, and a collapsed configuration which may represent the expandable member 114 being substantially deflated in order to not substantially occlude the blood vessel.

In some examples, the device 150 further includes at least one pressure sensor module (e.g., sensor module 117) that may be coupled to a distal end 118 of the expandable member 114. In some examples, at least one sensor module 156 is also or instead included on device 150. Sensor module 156 may be coupled to a portion of the inflation line 116 at or near to location 128. For example, the sensor module 156 may be positioned on the inflation line 116 upstream from the inflation port 120 (e.g., on the inflation line 116 toward the inflow end 102). The sensor modules 117, 156 may detect pressure in the blood vessel in which the device is installed, and the device can cause, based on the detected pressure, an increase or a decrease of the inflation fluid in the expandable member 114. In some examples, the sensed pressures/data from the sensor module 117 and the sensor module 156 are used together by the device to alleviate atrial pressure and/or renal pressure according. In some embodiments when one sensor module is used, the sensor module data may be used to estimate a desired outflow.

The device 150 may provide an additional advantage of allowing the expandable member 114 to intake fluid volumes beyond a fluid volume level indicated for a device that is bounded within a frame, such as frame 106. The advantage of being able to fill expandable member 114 to an increased fluid capacity can enable the device 150 to continue to occlude if the size of the SVC changes over time. In particular, the device 150 allows for adjustment of inflation fluid volume to compensate for blood vessel changes.

In some examples, the device 100, 150, 1300 may be a flow restrictor for a blood vessel that includes, the outer frame 106, 1320, one or more expandable members 114 or expandable annulus members 1350 where such members 114, 1350 may each include an inflation port (e.g., inflation port 120) coupled to a first portion (e.g., within a threshold distance of distal end 216b) of the inflation line 116 (or alternatively to separate inflation lines). Each expandable member 114, 1350 may define a volume configured to receive an inflation fluid therein through a respective inflation port 120. For example, the expandable member 114, 1350 may define a volume that may receive and hold inflation fluid from an actuation/pump device. However, in some embodiments, a pump is not included in the devices described herein (device 100, device 150, device 1300) and the inflation of particular reservoirs, balloons, expandable members, etc. occurs passively rather than actively pumping in inflation fluid. Note that particular reservoirs, balloons, expandable members, etc., of the invention may be pre-inflated to pre-set and/or fixed levels, e.g., during device deployment, depending on the particular embodiment.

The device 100, 150 may further include at least one anchor (e.g., anchor 126 of FIG. 1B) that may couple (e.g., secure, anchor, tether, or otherwise fasten) a second portion (e.g., location 128 of FIG. 1B) of the inflation line 116 to the device, e.g., to an internal surface of the outer frame 106, to allow the one or more expandable members 114 to oscillate within the outer frame 106 or within a portion of the blood vessel in response to blood flow through the blood vessel. For example, the one or more expandable members 114 may be within a threshold distance of distal end 118 of the inflation line 116. In particular, the expandable member 114 may be installed on line 116 at the threshold distance of about 1 mm to about 20 mm; about 1 mm to about 5 mm; about 5 mm to about 10 mm; about 10 mm to about 15 mm; or about 15 mm to about 20 mm from distal end 118 of the inflation line 116. The second portion (e.g., location 128 of FIG. 1B) of the inflation line 116 may be within a second threshold distance of the proximal end 108 of the outer frame 106. Put another way, the anchor 126 may be coupled to the inflation line 116 about 0 mm to about 70 mm; about 0 mm to about 10 mm; about 10 mm to about 20 mm; about 20 mm to about 30 mm; about 10 mm to about 20 mm; about 20 mm to about 30 mm; about 30 mm to about 40 mm; about 40 mm to about 50 mm; about 50 mm to about 60 mm; or about 60 mm to about 70 mm offset from the expandable member 114.

In some examples, the device 100, 150, 1300 includes at least one pressure sensor module (e.g., sensor module 117, sensors 1310, 1314). In some examples, the sensor module includes one or more sensors for sensing pressure, flow, or the like. In some examples, the sensor module includes one or more sensors for sensing pressure, flow, or the like, and additional electronics components such as hardware processor(s), processor(s), switches, flow meters, or the like, as described elsewhere herein. In some examples, the sensor module 117 may detect pressure in the blood vessel and, via a processor(s) and/or fluid pump(s) and/or valve(s), cause an increase or a decrease of inflation fluid in the expandable member 114 based on the detected pressure.

In some examples, the at least one sensor module 117 may be coupled to the distal end 216b of the inflation line and arranged within the SVC to measure (or sense) right atrial pressure. In some examples, the at least one sensor module 117 may be coupled to the inflation line 116 at a location 128 in which the sensor module 117 is arranged within a threshold distance of a proximal end 108 of the outer frame 106 and may detect SVC pressure or renal pressure from such a location.

In some examples, the device 100, 150, 1300 also includes a safety sensor module 125. The safety sensor module 125 may cause an interruption or stoppage of at least one cycle of the flow restricting of device 100, 150, 1300 in response to detecting a blood vessel pressure above a predefined safety threshold. The safety sensor module 125 may be placed at or near the proximal end 216a of inflation line 116 to detect right atrial pressure. An example predefined safety pressure threshold for right atrial pressure and/or SVC/renal pressure includes about 25 mmHg to about 35 mmHg; about 25 mmHg to about 30 mmHg; or about 30 mmHg to about 35 mmHg. The safety sensor module 125 may be placed at or near location 128 of inflation line 116 to detect SVC or renal pressure.

In some examples, the device 100, 150 is a device for modulating blood flow in a blood vessel which includes one or more expandable members having an inflation port (e.g., inflation port 120) coupled to an inflation line (e.g., inflation line 116) and an anchor having a first surface (e.g., surface 152) that may be coupled to the inflation line 116 along a portion of length of the inflation line 116 and a second surface (e.g., surface 154) that may be coupled to a wall of the blood vessel.

In some examples, the device 100, 150, 1300 further includes at least one pressure sensor module (e.g., sensor module 117, sensors 1310, 1314) that may be coupled to a distal end 118 of the expandable member 114, 1350. In some examples, the sensor module (e.g., sensor module 156) may be coupled to a portion of the inflation line 116, as shown in FIG. 1B. For example, the sensor module 156 may be positioned on the inflation line 116 upstream from the inflation port 120 (e.g., on the inflation line 116 toward the inflow end 102). The sensor modules 117, 156 may detect pressure in the blood vessel in which the device is installed, and the detected pressure value can be used to cause, using a processor and/or pump and/or valve, an increase or a decrease of the inflation fluid in the expandable member 114.

In some examples, the device 100, 150, 1300 includes an expandable member 114, 1350 that is coated with thromboresistant material. In some examples, the device 100, 150, 1300 includes an expandable member 114, 1350 that is coated with a Perylene micro-coating. In some examples, the device 100, 150, 1300 includes an outer frame 106, 1320 with an inner surface coated with thromboresistant material. For example, the coatings described herein may be any pro-endothelial factor including, but not limited to, endothelial growth factor, vascular endothelial growth factor, or any related compound. Example thromboresistant materials that may coat the expandable members, frames, inflation lines, or the like may a polymer (e.g., silicones, poly (urethanes), poly (acrylates), or copolymers such as poly(ethylene vinyl acetate), an anti-thrombogenic drug, a textile (e.g., woven, knitted, nonwoven, or braided), tissue (e.g., bovine pericardium, equine pericardium, porcine vena cava, etc.), or a combination thereof.

FIG. 2A illustrates an embodiment of a device 200 for modulating blood flow implanted in a blood vessel and electrically coupled to a control device 210. In this example, the device 200 may represent the flow modulating device 100, the flow modulating device 150, the flow modulating device 1300, or another flow modulating device or schematic representation, as described herein. As shown, the device 200 is implanted in a left brachiocephalic vein. While device 200 is shown implanted in vein, the device may alternatively be implanted along the SVC, or another vessel depicted in FIG. 2A.

The device 200 may include a frame 206, which may be a stent frame (similar to frame 106 or frame 1320) or an anchor frame (similar to anchor 126). The frame 206 includes an inflow end 202 and an outflow end 204. The device 200 may further include an inflation control device 210 coupled to an inflation line 216 that is further coupled to an expandable member 214. The expandable member 214 is depicted in a collapsed configuration at member 215 and in an expanded configuration at member 217, shown here in dashed line to indicate a different state of member 214 than the state shown by member 215. While both states are depicted in FIG. 2A, a single state (i.e., any single state between collapsed and fully expanded) is selectively activated based on sensed information or manual activation of member 214. In some examples, the device 200 further includes one or more recapture features, as described for example with respect to FIGS. 12-13. In some examples, the one or more expandable members of device 200 may be one or more expandable annulus members. The one or more expandable annulus members may include one or more inflatable inner cavities, as described with respect to FIGS. 13-15.

The inflation control device 210 includes a reservoir 212, a pump 213, and one or more controls (not shown). In general, the reservoirs described herein may include or otherwise contain inflation fluid. For example, the reservoirs may include walls, ends, or portions that are sealed or at least partially scaled to receive and hold a volume of inflation fluid. The pump 213 can be a motorized pump (e.g., a positive-displacement pump, an impulse pump, a velocity pump, a steam pump, a valveless pump, and/or other pump using electrical or inductive power), a manual pump, for example like a syringe, or a manual pump that is responsive to anatomical changes (e.g., diaphragm position during breathing, rib cage expansion and contraction, etc.). The pump 213 acts upon reservoir 212 to cause inflation fluid to flow into the inflation line 216. For example, the inflation line 216 may be coupled to the reservoir 212 and pump 213 (or alternatively to a subcutaneously implanted reservoir and/or pump) and actuation of the pump 213 may cause inflation fluid from the reservoir to be pumped into the expandable member 214 to cause partial or full inflation of the expandable member 214. Similarly, inflation fluid may be pumped from the expandable member 214 back to the reservoir 212 by the pump 213, for example, to cause partial or full deflation of the expandable member 214. Alternatively, the inflation fluid may be expelled into the anatomy to deflate the expandable member 214, for example in the case of saline.

An optional valve (manual or motorized valve) can be actuated to allow inflation fluid to flow through inflation line 216 or inhibit inflation fluid to flow through inflation line 216. The optional valve may be a one-way valve (e.g., a check valve) or a multi-way valve (e.g., including two or more ports). A second end 216b of inflation line 216 is fluidly connected to an inflation port of at least one expandable member 214, which may be coupled to at least a portion of a frame 206 (or anchor) of flow modulating device 200. The inflation fluid may flow into a volume defined by an expandable member 214. The inflation fluid may fill at least a portion of the volume defined by the expandable member 214 to cause the expandable member 214 to reversibly inflate and at least partially occlude a lumen of the frame 206 of the flow modulating device 200, which may at least partially occlude a vessel in which the device 200 is positioned. In some examples, the device 200 may at least partially occlude the vessel in the embodiment in which the expandable member 214 is anchored to the vessel. The inflation fluid can include a gas, saline, heparinized saline, blood, contrast, and the like, or a combination thereof.

In some examples, the inflation line 216 is coupled to a subcutaneously implanted reservoir and pump and in such examples, actuation of the implanted pump 213 may cause inflation fluid from the implanted reservoir to be pumped into the expandable member 214 to cause partial or full inflation of the expandable member 214, as shown by expandable member 217. The reservoirs and pumps described herein may further include or be coupled to a power source. For example, a power source, such as a battery 223 may be coupled to the pump 213.

The frame 206 may further include an anchor, sleeve, patch, attachment device, coupler, or the like (e.g., anchor 126). In some examples, the anchor may be a sleeve for receiving the inflation line 216 therethrough. The sleeve may include an inner surface and an outer surface. The inner surface may receive and hold the inflation line 216 in a predefined location along the inflation line 216. The outer surface of the sleeve may be arranged to attach to the frame 206. In examples without a frame, the outer surface of the sleeve may be arranged to attach to the blood vessel wall or other implanted device within the vessel. In general, the anchor 126 may tether the inflation line 216 (attached to the expandable member 214) to a location along the frame 206. The inflation line 216 may be flexible to enable the expandable member 214 to move (e.g., oscillate) within the blood vessel (or within the frame 206) in response to blood flow through the blood vessel. In the example in which the blood vessel is the SVC, the outer frame 206 may be implanted in a vessel branching from the SVC, such as the left brachiocephalic vein, which may allow the expandable member 214 to be tethered to the outer frame 206 at a length such that the expandable member 214 extends beyond an end perimeter of the outer frame 206 and into the SVC substantially adjacent to an azygous junction. By allowing the expandable member 214 to extend beyond the frame 206 and near to the azygous junction may allow the expandable member 214 to substantially block the azygous junction when the expandable member 214 is in an expanded configuration, as shown by expandable member 217. Similarly, the azygous junction may be substantially unblocked when the expandable member 214 is in a collapsed or deflated configuration, as shown by expandable member 215. In some examples, the blood vessel is a vena cava such as the IVC.

FIG. 2B illustrates an embodiment of the device 200 for modulating blood flow implanted in a blood vessel and electrically coupled to the inflation control device 210. In this example, device 200 is shown positioned in the SVC 250 of a heart 252. Although the flow modulating device 200 is shown as positioned in an SVC 250, one of skill in the art will appreciate that a flow modulating device, such as any of the flow modulating devices described herein or portions thereof may be positioned in any bodily lumen or vessel (e.g., VC, IVC, SVC, renal artery, renal vein, etc.) to regulate flow therethrough or through an adjacent vessel fluidly connected to the vessel in which the device is positioned.

The flow modulating device 200 may include the frame 206 having an inflow end and an outflow end. The device 200 may include a skirt membrane 122, as described in elsewhere herein. The frame 206 defines a lumen including an internal surface between the inflow end and the outflow end. One or more expandable members (e.g., expandable members 114, 214; expandable annulus members 1350) are coupled to at least one portion of the internal surface of the frame, as shown in FIGS. 1A-2A. The one or more expandable members, or an inner cavity of an expandable member, each define a volume for receiving inflation fluid therein through an inflation port (e.g., inflation port 120) to inflate the one or more expandable members.

In some embodiments, the device 200 may include a skirt membrane positioned as shown by skirt membrane 122 in FIG. 2B at a distal end of frame 206. In some embodiments, the device 200 may include a skirt membrane (such as skirt membrane 122) positioned at a medial location along the frame 206. In some embodiments, the device 200 may include a skirt membrane (such as skirt membrane 122) positioned at an opposite side of the frame 206 (e.g., a proximal end of frame 206). In some embodiments, the device 200 may be positioned in the SVC 250 at another location along the SVC 250.

The one or more expandable members (e.g., expandable member 214, expandable member 114, expandable annulus member 1350) may be each reversibly inflatable to a plurality of inflation configurations (e.g., states) to partially or fully occlude the lumen of the frame. For example, a volume defined by an expandable member, in an unrestricted or deflated/collapsed configuration, may be empty or have substantially no inflation fluid in the volume defined by the expandable member. Further, for example, a volume defined by an expandable member, in a restricted or expanded configuration, may include inflation fluid in the volume and/or be substantially full of an inflation fluid in the volume defined by the expandable member. Still further, for example, in an intermediate or partially expanded configuration, a volume defined by the expandable member may be partially full (or partially empty) such that an inflation fluid is used to partially fill the volume defined by the expandable member.

An example threshold level of inflation for one or more expandable members (e.g., expandable member 214, expandable member 114, one or more expandable annulus member 1350) may include about 40% to about 80% of full occlusion of a respective expandable member; about 40% to about 45%; about 45% to about 50%; about 50% to about 55%; about 55% to about 60%; about 60% to about 65% of full occlusion of the respective expandable member; about 65% to about 70% of full occlusion of the respective expandable member; about 70% to about 75% of full occlusion of the respective expandable member; or about 75% to about 80% of full occlusion of the respective expandable member.

Although a collapsed configuration, a partially expanded configuration, and an expanded configuration are described, one of skill in the art will appreciate that any number of intervening flow configurations/states between the aforementioned configurations is also possible and contemplated herein.

The inflation line 216 may be flexible and may allow from atraumatic tracking. In addition, the inflation line 216 may be length adaptable to allow the length to be adjusted according to an anatomy of the blood vessel or the subject in which the device 200 is to be implanted. The inflation line 216 may be coupled to the inflation control device 210 via a barb or Luer® connection.

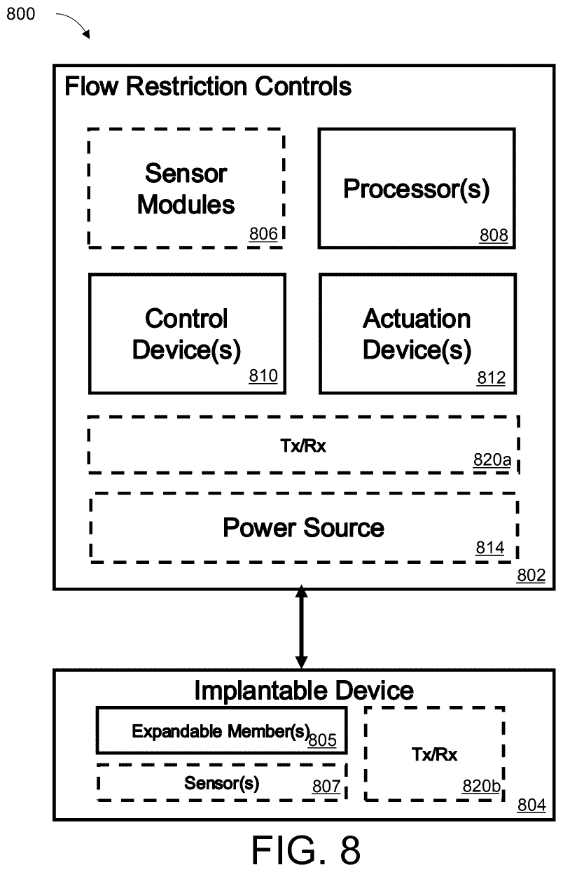

In some examples, as shown in FIG. 8, the device 200 may further include one or more sensor modules 806 (e.g., a sensor module 117 and/or a sensor module 156) for detecting a pressure in the blood vessel, a microprocessor (e.g., processor 808) electrically coupled to the sensor modules 806, and/or a power source (e.g., power source 814) electrically coupled to an actuator (e.g., actuation device 812) associated with device 200, the microprocessor 808, and/or the sensor modules 806. For example, the sensor modules 806 may sense characteristics of blood flow in the blood vessel (e.g., blood pressure) and may provide corresponding pressure data to the microprocessor 808, with the microprocessor 808 adapted to process the pressure data and provide signals to the actuator (e.g., actuation device 812) to trigger expansion/contraction of the expandable member 214. In operation, the microprocessor 808 can receive a signal from the sensor modules 806 that is indicative of a pressure in the blood vessel. The microprocessor 808 can process the signal and generate and provide a control signal (e.g., via control devices 810) to trigger expansion or reduction of expandable member 214 based on the sensed pressure in the blood vessel.

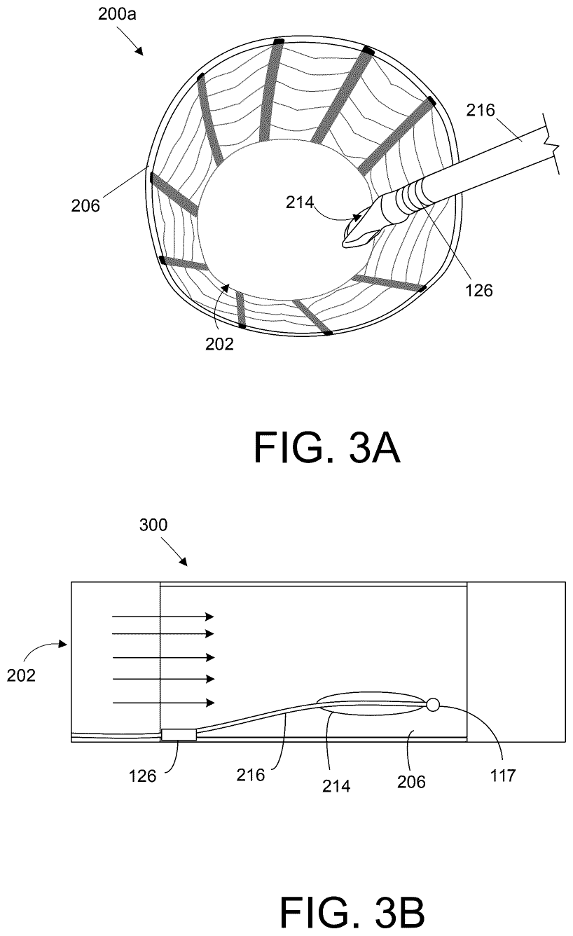

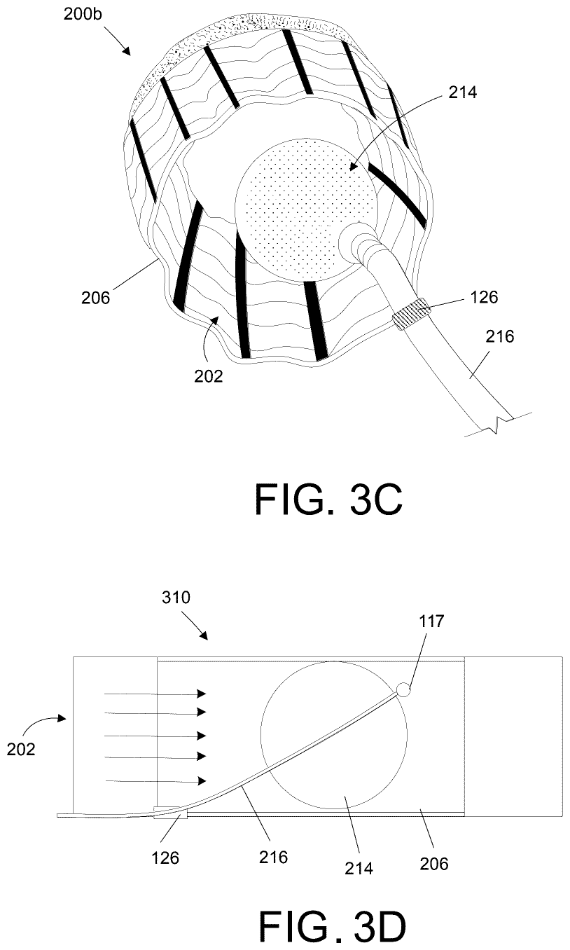

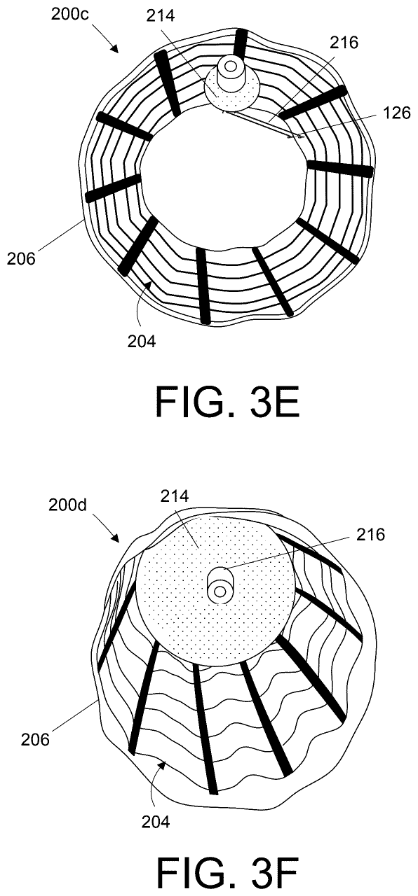

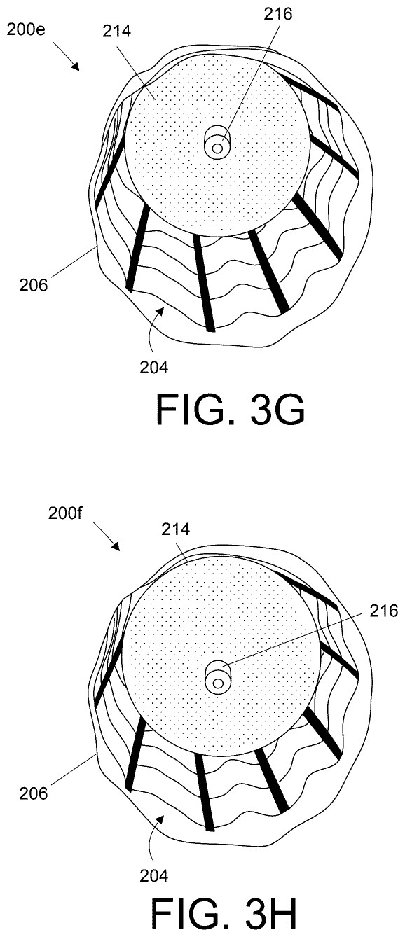

FIG. 3A illustrates a bottom-up perspective view of the example flow modulation device 200a in a deflated state. In this example, the device 200a may represent the flow modulating device 200, the flow modulating device 100, the flow modulating device 150, or another flow modulating device or schematic representation, as described herein. The device 200a includes a deflated expandable member 214 (e.g., a balloon) tethered to the inflation line 216 and substantially coupled to frame 206 by anchor 126. As shown, the cavity of expandable member 214 is in fluid communication with the inflation line 216 and is otherwise unattached to the frame 106. The inflation line 216 extends proximally from and coupled to the expandable member 214 and is coupled by anchor 126 to one side of the frame 206. While the anchor 126 is depicted on a side of the frame 206, the anchor may alternatively be arranged and coupled to another location within or on a permitter of frame 206. The device 200a can be implanted in the SVC, for example, such that when the expandable member 214 is deflated (e.g., collapsed), as shown in FIG. 3A, blood can flow through the device 200a into the right atrium. When the expandable member 214 is inflated (e.g., expanded or partially expanded) as shown in FIG. 3D, blood flow is blocked or partially blocked, respectively.

FIG. 3B illustrates a schematic representation 300 of the anchored flow modulation device 200a in a deflated state. The device 200a includes the expandable member 214 coupled to the inflation line 216. The inflation line 216 may be in fluid communication with a subcutaneous reservoir and pump, as shown in FIGS. 2A, 2B. In this example, the expandable member 214 is shown deflated in the collapsed configuration, which allows oscillation of expandable member 214 within the frame 206 when blood flows from the inflow end 202. The representation 300 includes a sensor module 117 arranged on a distal end of the expandable member 214. The sensor module 117 may detect pressure in a surrounding environment of device 200a.

FIG. 3C illustrates a bottom-up perspective view of the device 200a shown in FIG. 3A in an example partially expanded configuration depicted as device 200b. In this example, the device 200b may represent the flow modulating device 200, the flow modulating device 100, the flow modulating device 150, or another flow modulating device or schematic representation, as described herein. The device 200b includes a partially expanded expandable member 214 (e.g., a balloon) tethered to the inflation line 216 and substantially coupled to frame 206 by anchor 126. As shown, the cavity of expandable member 214 is in fluid communication with the inflation line 216 and is otherwise unattached to the frame 106. The inflation line 216 extends proximally from and coupled to the expandable member 214 and is coupled by anchor 126 to one side of the frame 206. While the anchor 126 is depicted on a side of the frame 206, the anchor may alternatively be arranged and coupled to another location within or on a permitter of frame 206. The device 200b can be implanted into the SVC, for example, such that when the expandable member 214 is deflated (e.g., collapsed), as shown in FIG. 3A, blood can flow through the device 200b into the right atrium.

FIG. 3D illustrates a schematic representation 310 of the anchored flow modulation device 200b in an expanded state. The device 200b includes the expandable member 214 coupled to the inflation line 216. The inflation line 216 may be in fluid communication with a subcutaneous reservoir and pump, as shown in FIGS. 2A, 2B. In this example, the expandable member 214 is shown expanded in a fully expanded configuration, which allows one or more surfaces of an outer portion of member 214 to use frame walls to at least partially block blood flow from the inflow end through the lumen of the device 200b. The representation 310 includes a sensor module 117 arranged on a distal end of the expandable member 214. The sensor module 117 may provide sensed pressure data to trigger actuation of device 200b based on sensed pressure. For example, actuation of device 200b may be responsive to elevated pressure sensed by one or more sensors (e.g., sensor module 117). Actuation of device 200b may include pumping inflation fluid from the reservoir into the expandable member 214 to expand the member 214 (as shown in FIG. 3D) and alleviate the sensed pressure or alleviate pressure downstream from the sensed pressure. For example, upon detecting (e.g., sensing by sensor module 117 or another sensor) pressure exceeding a predefined threshold described elsewhere herein, the device 200b may be actuated to expand (e.g., inflate) expandable member 214 to constrict or block blood flow through the lumen of device 200b (and in the example of FIG. 2B), thereby constricting or blocking blood flow through the SVC.

FIG. 3E illustrates a top-down perspective view of the device 200c shown in FIG. 3A in a deflated configuration. The device 200c is depicted from the outflow end 204 with the expandable member 214 coupled to inflation line 216 and in a fully collapsed configuration.

FIG. 3F illustrates a top-down perspective view of the device 200d shown in FIG. 3A in an example partially expanded configuration. The device 200d is depicted from the outflow end 204 with the expandable member 214 coupled to inflation line 216 and in a partially expanded configuration.

FIG. 3G illustrates a top-down perspective view of the device shown in FIG. 3A in another example partially expanded configuration. The device 200e is depicted from the outflow end 204 with the expandable member 214 coupled to inflation line 216 and in yet another example partially expanded configuration.

FIG. 3H illustrates a top-down perspective view of the device shown in FIG. 3A in a fully expanded configuration. The device 200f is depicted from the outflow end 204 with the expandable member 214 coupled to inflation line 216 and in a fully expanded configuration.

By way of example, in the depicted position in FIG. 3D, the expandable member 314 may be expanded to ensure that no blood may flow from the SVC into the right atrium, thus reducing right atrium total blood volume. Such a flow modulation may result in right atrial pressure reduction. A reduced right atrial pressure can, in turn, result in a larger pressure difference on one or more renal vessels, which may cause an increase in the evacuation of fluids through the renal vessels. When the sensor module 117, for example, senses that pressure is sufficiently decreased to a value below a predefined threshold described elsewhere herein, the device 200 may trigger deflation or collapsing of expandable member 114 by drawing (e.g., extracting, transferring, etc.) inflation fluid back into the reservoir 212, thus allowing for renewed blood flow into the right atrium (as shown in FIG. 3A). This process may repeatedly and/or iteratively cycle between the collapsed configuration (e.g., FIGS. 3A, 3E) and the expanded configuration (e.g., FIG. 3H) and/or another configuration (e.g., partially expanded configuration FIGS. 3F, 3G) in response to the sensor module 117 (or another sensor module or sensor) detecting right atrial pressures above the predefined thresholds described elsewhere herein.

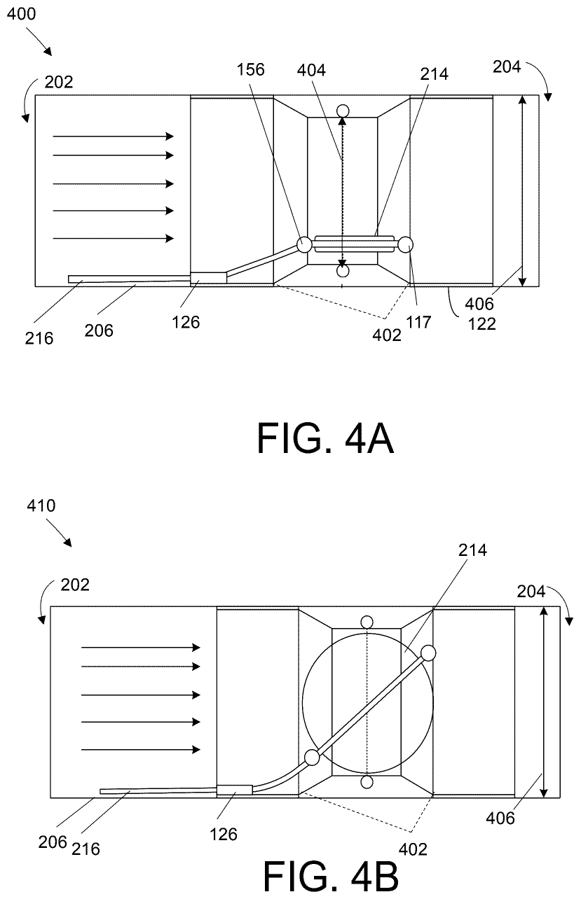

FIG. 4A illustrates another schematic representation 400 of an anchored flow modulation device in a deflated/collapsed configuration. For example, the device in FIG. 4A may represent device 100, device 150, or device 200. As shown, the frame 206 anchors the inflation line 216 at anchor 126 and allows oscillatory movement of the expandable member 214. Sensor modules 117 and/or 156 may be optionally installed on one or more of the expandable members 214 and the inflation line 216. The expandable member 214 in FIG. 4A is depicted in a collapsed configuration.

In this example, the frame 206 may have a lumen that has a variable cross section through the length of the outer frame 206. For example, the lumen may be radially narrowed along an intermediate portion 402 located between the inflow end 202 and the outflow end 204. The intermediate portion 402 may be sized in a portion of the lumen to contain (or constrain when fully expanded) the expandable member 214 to a predefined diameter 404, for example, because the intermediate portion 402 may have an annular cross section that is less than an annular cross section at the inflow end 202 or the outflow end 204. While the intermediate portion 402 narrows the lumen of device 200, passage of a catheter and/or other devices is still possible when the expandable member 214 is deflated.

The smaller cross section of the intermediate portion 402 allows the use of a smaller balloon size than would be used with a uniform cross section from inflow end 202 to outflow end 204. The smaller balloon size provides an advantage of using less fluid and therefore smaller inflation fluid reservoirs for the device 200 than would be used with a uniform cross section from inflow end 202 to outflow end 204. Smaller components can enable improved insertion and function. Such components may also be deployed less often to alleviate pressure if the narrowness of the intermediate portion 402 causes a built-in stepped downsizing of flow capacity through the lumen of device 200, which may reduce blood flow a lesser amount than the occlusion caused by the expandable member 114 when expanded, but the lesser amount of blood may alleviate enough pressure to allow the device 200 to rest or not operate as often while still maintaining pressure levels for the subject in which device 200 is implanted.

In addition, having a device with an intermediate portion 402 that is narrower than end (inflow/outflow) sections provides an additional advantage of removing the unknown parameter of exact vessel diameter 406 because the narrowed intermediate portion 402, for example, can be designed to function with an expandable member 214 that is designed to expand to occlude blood flow through an exact and known diameter of the intermediate portion rather than relying on sensors and vessel diameter margins (e.g., based on a vessel diameter 406) of error to ensure that the expandable member 214 is fully expanded. For example, the narrowed lumen caused by intermediate portion 402 enables the expandable member 214 to be selected for a substantially maximum or full expansion defined by a diameter 404 of the narrowed lumen. The diameter may be about 17 mm to about 18 mm; about 17 mm to about 17.3 mm; about 17.3 mm to about 17.6 mm; or about 17.6 mm to about 18 mm at the intermediate portion 402.

FIG. 4B illustrates another schematic representation 410 of the anchored flow modulation device 200 in an expanded configuration. Similar to FIG. 4A, the frame 206 anchors the inflation line 216 at anchor 126 and allows oscillatory movement of the expandable member 214. Sensor modules 117 and/or 156 may be optionally installed on one or more of the expandable members 214 and the inflation line 216. The narrowed intermediate portion 402 is again shown. The expandable member 214 in FIG. 4B is depicted in an expanded configuration to fill the intermediate portion 402 and occlude blood flow through the lumen of device 200.

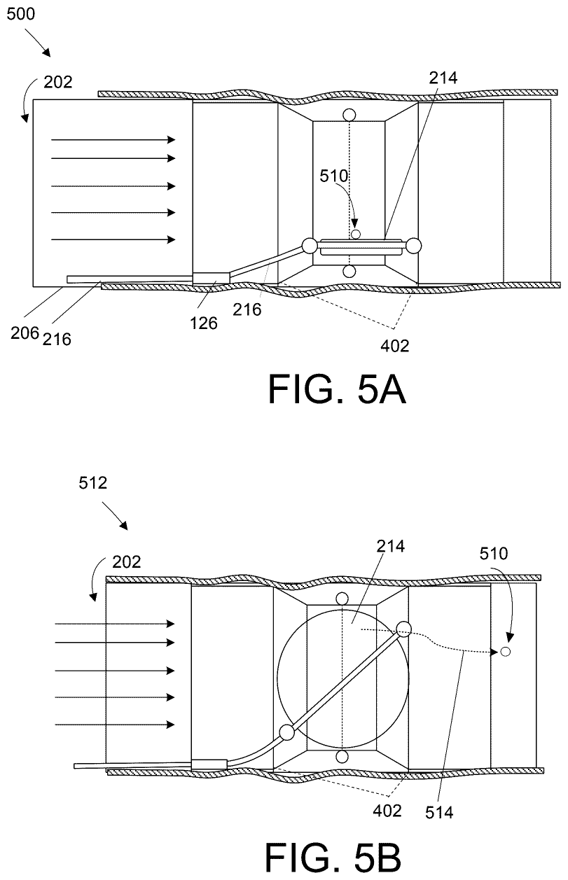

FIGS. 5A-5B illustrate schematic representations of the example device of FIGS. 4A-4B within a blood vessel. FIG. 5A depicts a schematic representation 500 of device 200 with a narrowed intermediate portion 402 containing the expandable member 214 in the collapsed configuration. In this example, the expandable member 214 is anchored via inflation line 216 by anchor 126 to the frame 206, but the expandable member 214 is free to oscillate or otherwise move and change positions (e.g., side-to-side movement) within the device and frame when inflated and/or deflated. With the expandable member 214 able to oscillate or otherwise move about, blood cells and other small particles 510 are prevented from building up on the member 214.

FIG. 5B depicts a schematic representation 512 of device 200 with the narrowed intermediate portion 402 containing the expandable member 214 in a fully expanded configuration. Because the device 200 of FIG. 5A is able to oscillate or otherwise move about within the frame 206, and/or also due to expansion/contraction (e.g., elongation/shortening) of the expandable member 214, blood cells and/or other small particles 510 that might otherwise mass together are likely to be shed from the expandable member prior to massing together, as shown by arrow 514.

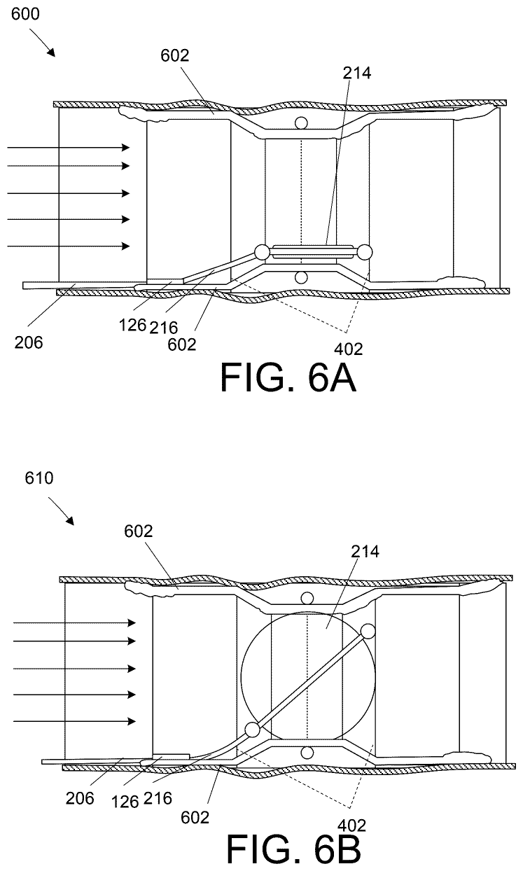

In addition, one or more coatings may further be included on device 200 to further reduce the chance of tissue build-up (e.g., from blood cells, fat, etc.) on the device. FIGS. 6A-6B illustrate schematic representations of the example device of FIGS. 4A-4B including an inner surface coating. FIG. 6A is a schematic representation 600 of the anchored flow modulation device 200 in a deflated position. FIG. 6A depicts device 200, for example, with a narrowed intermediate portion 402 and a coating 602 along the inner surface of frame 206. As shown, the frame 206 anchors the inflation line 216 at anchor 126 and allows oscillatory movement of the expandable member 214. The expandable member 214 in FIG. 6A is depicted in a collapsed configuration.

FIG. 6B illustrates another schematic representation 610 of the anchored flow modulation device 200 in an expanded configuration. Similar to FIG. 6A, the frame 206 anchors the inflation line 216 at anchor 126 and allows oscillatory or other movement of the expandable member 214 within the frame 206. The narrowed intermediate portion 402 is again shown. The expandable member 214 in FIG. 6B is depicted in an expanded configuration to fill the intermediate portion 402 and occlude blood flow through the lumen of device 200. In this example, the coating 602 may prevent re-endothelization of the inner surface of the device 200 to ensure that full closure (e.g., with the expandable member 214 in the fully expanded configuration) of the flow passage through the lumen of device 200 can continue to function over time, as the device 200 may be implanted for one or more years.

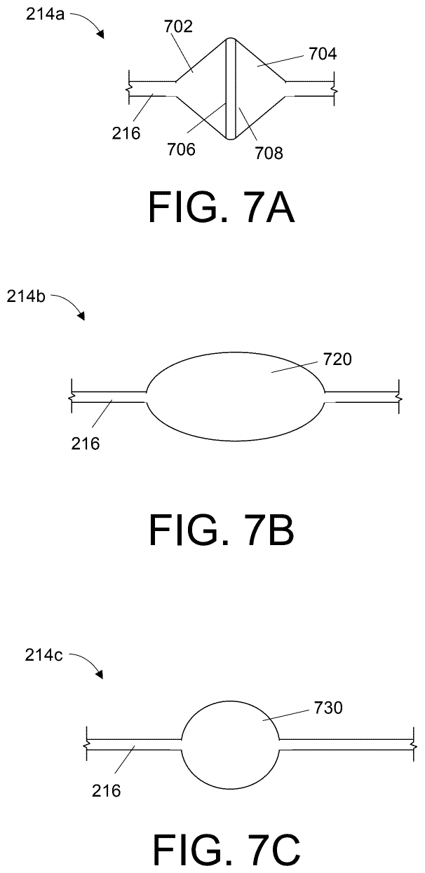

FIGS. 7A-7C illustrate example expandable members for use with any of the devices described herein. Various shapes of expandable members 214 may be used with the devices described herein. In some examples, multiple members 214 may be used and each member may be of a same or a different shape.

FIG. 7A depicts an example expandable member 214 shown here as expandable member 214a. Expandable member 214a is shown as a substantially biconical shape with two substantially congruent cones 702, 704 joined at respective bases 706, 708. The expandable member 214a may be configured to inflate up to a substantially maximum elongation of about 300 percent. For example, the expandable member 214a may receive fluid from reservoir 212 and pump 222 through inflation line 216 to fill a volume of expandable member 214a up to a substantially maximum elongation of about 300 percent. In some examples, the expandable member 214a is a single conical structure rather than a biconical structure. The biconical or conical shape may allow for the use of less inflation fluid which may limit elongation and improve stretch-fatigue resistance on the material that forms the expandable member 214a.

In some examples, the expandable member 214a may be inflated to elongate from about 25 percent to about 350 percent; about 50 percent to about 100 percent; about 100 percent to about 150 percent; about 150 percent to about 200 percent; about 200 percent to about 250 percent; about 250 percent to about 300 percent; or about 300 percent to about 350 percent.

FIG. 7B depicts an example expandable member 214 shown here as expandable member 214b. Expandable member 214b is shown as a substantially elongated spherical shape in which fluid may enter a volume 720. For example, the expandable member 214b may receive fluid from reservoir 212 and pump 222 through inflation line 216 to fill the volume 720.

FIG. 7C depicts an example expandable member 214 shown here as expandable member 214c. Expandable member 214c is shown as a substantially spherical shape with a substantially annular cross section when in an expanded position. The expandable member 214c may be configured to inflate up to a substantially maximum elongation of about 900 percent. For example, the expandable member 214c may be receive fluid from reservoir 212 and pump 222 through inflation line 216 to fill the volume 730 of expandable member 214a up to a substantially maximum elongation of about 900 percent. In some examples, the expandable member 214a may expand from about zero percent to about 900 percent; about 100 percent to about 200 percent; about 200 percent to about 300 percent; about 300 percent to about 400 percent; about 400 percent to about 500 percent; about 500 percent to about 600 percent; about 600 percent to about 700 percent; about 700 percent to about 800 percent; or about 800 percent to about 900 percent.

While the example expandable members described herein (e.g., expandable members 114, 214, 214a, 214b, 214c, 215, 217) include the use of a single member for occluding a lumen or blood vessel, any number of expandable members can be used (e.g., one, two, three, four, five, one to ten, greater than ten, etc.).

FIG. 8 is a block diagram of an example system 800 for modulating blood flow through one or more blood vessels. The system 800 may be used with any of the flow restricting devices described herein (e.g., device 100, 150, 200, 1300). As shown, the system 800 includes flow restriction controls 802 and at least one implantable device 804 that includes one or more expandable members 805 that may modulate blood flow by increasing or decreasing fluid held within the one or more expandable member(s) 805. The implantable device 804 may correspond to any of the flow restricting devices described herein such as device 100, device 150, device 200, etc.

The flow restriction controls 802 may include one or more optional sensor modules 806 (e.g., sensor module 117 and/or sensor module 156 and/or sensor module 125 and/or sensor 1310, and/or sensor 1314) coupled to one or more sensors 807, one or more processors 808, one or more control devices 810, and one or more actuation devices 812, or any combination thereof that may increase or decrease fluid in the one or more inflatable members 805. Optionally, the flow restriction controls may include a power source 814 that may be internal to the controls 802, internal to the implantable device 804, or external to both the flow restriction controls 802 and the implantable device 804. In some examples, the power source 814 may be electrically wired to flow restriction controls 802 and/or implantable device 804.

In some examples, the power source 814 may be remotely accessed (e.g., wirelessly) by flow restriction controls 802 and/or implantable device 804.

The optional sensors 807 and/or sensor modules 806 may generally function to sense (e.g., detect) properties of the blood in which the sensor(s) are disposed within. For example, the optional sensors 807 and/or sensor modules 806 may detect blood pressure within the blood vessel and/or any other physiological or anatomical parameters or properties of the blood or vessel. The optional sensors 807 and/or sensor module 806 may include one or more of an image sensor, a strain gauge, a piezoelectric sensor, a capacitance sensor, and/or a vacuum pressure sensor alone or in combination with a processor, such as processor 808. In general, sensor signals from sensors/sensor module 806 may be transmitted to control devices or elements described herein via a wired or wireless connection. Additionally, and optionally, the sensors 807 and/or sensor modules 806 may be coupled to and utilize one or more processors 808 to transmit data to remote computing devices. The transmitted data may include sensor measurements, device position data and/or statistics, actuation events, or any other data from the system 800, devices 100, 150, 200, 1300, or another device communicably coupled to processors 808.

In some examples, the optional sensors 807 may be located within optional sensor modules 806. In some examples, the optional sensors 807 may be located on or near implantable device 804, inflatable member 805, and/or inflation line associated with device 804. Optional transmit and receive hardware 820a, 820b may be included in either or both of flow restriction controls 802 and/or implantable device 804 in order to send and receive commands, measurements, or other data amongst controls 802 and device 804 and/or another computing device in wireless communication with either or both controls 802 and device 804. The optional transmit and receive hardware 820a, 820b may include one or more wireless transceivers, wireless receivers, wireless transmitters, antennas, or the like to monitor and/or control device 804 operation.

The processors 808 may include one or more microprocessors, microcontrollers, or the like, as described elsewhere herein. In some examples, the devices described herein may be expanded or deflated using one or more control devices 810. The control devices 810 may include active or passive controls including, but not limited to wires, sutures, operated switches, motor controllers, and/or antennas. In some examples, the control devices 810 may include external control devices including, but not limited to remote computers, tablets, smart phones, and/or external control devices for powering and/or controlling the flow restriction controls 802.

In some examples, the devices described herein may be expanded or deflated using one or more actuation devices 812. The actuation devices 812 may include mechanically actuating devices, electrically actuated devices, electromechanically actuated devices, or a combination thereof. For example, actuation devices 812 may include any one or more of a wire, a suture, a pull wire, a linear actuator (e.g., a pneumatic linear actuator, an electromechanical linear actuator, or a hydraulic linear actuator), a magnet or coil, etc. The power sources 814 may include, but are not limited to, battery power, wall power, magnets, induction coils, or the like.

In operation of system 800, the actuation device 812 may be coupled to the control device 810, which may manipulate or move portions of the implantable device 804 based on one or more signals received from a sensor module 806. In embodiments that utilize a processor 808, the processor 808 may be communicatively coupled to sensor modules 806, control devices 810, actuation devices 812, power source 814, and/or implantable device 804 to actuate the implantable device 804 into a restricted blood flow state, an unrestricted blood flow state, or any position therebetween. The processor 808, of some embodiments, may be or integrated into sensor modules 806.

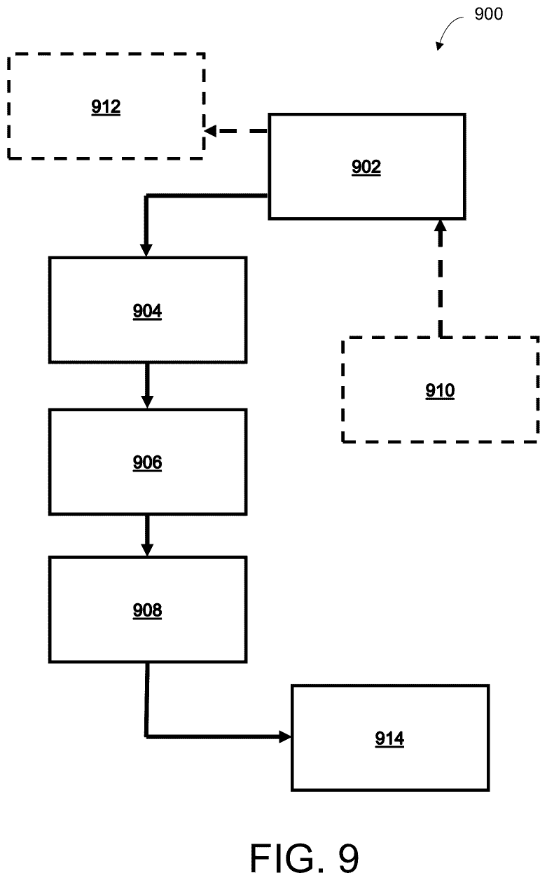

FIG. 9 is a schematic diagram of an example embodiment of a system 900 for modulating blood flow through a blood vessel. In some examples, the system 900 may include a first magnet 906, an actuation device 908 (e.g., inflation control device 210), and a control element 914. The first magnet 906 may be operatively coupled to the actuation device 908. The actuation device 908 may be operatively coupled to the control element 914 to effect movement of the control element 914. In some examples, the control element 914 is a pump (e.g., pump 213). In some examples, the control element 914 is a membrane. In some examples, the control element 914 is a control wire. In some examples, the control element 914 is a catheter portion.

In some examples, the system 900 may provide a manual or a power actuated way of expanding or deflating the expandable members described herein (e.g., expandable members 114, 214, 805). For example, the devices 100, 150, 200, or 1300 may include the actuation device 908 that may be manually controlled or powered and controlled (e.g., by control element 914) to trigger expansion or deflation of the expandable member(s) 805.

In some examples, the system 900 may include a control device 902 operatively coupled to a second magnet 904. The control device 902 can include a microprocessor, power source (a battery, a capacitor, wall outlet, or any other suitable power source), antenna, operated switches, and/or any other control devices as described in FIG. 8. The control device 902 and second magnet 904 may be located externally, but proximal to a subject in which the system 900 is implanted. In some examples, the control device 902 and second magnet 904 may be implanted (e.g., subcutaneously, intravascularly, etc.) in the subject. In some examples, both the first magnet 906 and the second magnet 904 may be permanent magnets. In some examples, the first magnet 906 is a permanent magnet and the second magnet 904 is an electromagnet.

Optionally, the system 900 may include one or more sensors/sensor modules 910 (e.g., sensor module 117, sensor module 156, sensor module 125, sensors 807, sensor 1310, sensor 1314, etc.). The sensors/sensor modules 910 may sense one or more physiological or anatomical attributes and output a signal to the control device 902, which may output an activation signal to the actuation device 908 to tension or release tension (e.g., cause intake or outtake of fluid from expandable member 805) based on movement of the control element 914.

The second magnet 904, although external to the subject or implanted at a second location (the implantable device being at a first location), may be placed operationally proximal to the first magnet 906. By doing so, the magnetic pole orientation of the second magnet 904 influences the magnetic pole direction of the first magnet 906. For example, a magnetic gear train may be generated between the second magnet 904 and the first magnet 906, such that when the control device 902 rotates the second magnet 904, the first magnet 906 is rotated in an opposing direction. Rotating the first magnet 906 induces movement in the actuation device 908, which tensions or releases tension (e.g., causes intake or outtake of fluid from expandable member 805) based on movement of the control element 914 or moves the control element 914 to a restricted (e.g., partially or fully expanded) or unrestricted (e.g., deflated, collapsed) blood flow state (e.g., configuration), respectively.

The control device 902 may receive signals from one or more optional sensors 910. Such signals may be indicative of characteristics of blood flow in the blood vessel (e.g., blood pressure). For example, when the control device 902 receives a signal indicative of a measured pressure higher than a predefined level, the control device 902 can cause the second magnet 904 to rotate. The rotation of the second magnet 904 can cause the first magnet 906 to rotate, which may actuate the actuation device 908 to move the control element 914 towards a partially or fully expanded configuration. Further, when the control device 902 receives a signal indicative of a measured pressure lower than the predefined level, the control device 902 may cause the second magnet 904 to rotate in an opposing direction. The rotation of the second magnet 904 causes the first magnet 906 to rotate, thereby actuating the actuation device 908 to move the control element 914 into the collapsed configuration.

In general, sensor signals from sensors 910 may be transmitted to control devices or elements described herein via a wired or wireless connection. Additionally, and optionally, the sensors 910 may utilize one or more processors to transmit data to remote computing devices. The transmitted data may include sensor measurements, device position data and/or statistics, actuation events, or any other data from the system.

FIGS. 12-14 illustrate an embodiment of a device 1300 for modulating blood flow in a blood vessel. FIG. 12 illustrates the device 1300 including an outer frame 1320 defining an inner surface 1365 and one or more recapture features 1330a, 1330b, 1330c, 1330d, 1330c, 1330f, and/or 1330g. Although a plurality of recapture features 1330a-1330g are shown, one of skill in the art will appreciate that any number of recapture features may be used, for example, one or more, more than one, at least one, or a plurality of recapture features. The recapture features 1330a-1330g may reversibly interact with one or more complementary features (e.g., aperture, slot, groove, etc.) on a delivery system. As such, when in the sheath or the catheter, the one or more recapture features 1330a-1330g may be secured in the one or more complementary features on the delivery system. When the sheath or catheter is pulled back or removed from the device 1300 during deployment, the one or more recapture features 1330a-1330g may be released from the one or more complementary features of the delivery system to enable device 1300 expansion. Further, in some embodiments, the one or more recapture features 1330a-1330g may be radiopaque. Although a droplet shape is shown for the tip of the one or more recapture features 1330a-1330g, one of skill in the art will appreciate that any shape may be used to perform the intended function, and the shape may be selected based on performance (e.g., smooth deployment and/or recapture). For example, the one or more recapture features 1330a-1330g may be circular, square, ellipse, flag-shaped, etc.

As shown in FIG. 12, device 1300 may further include an inflow sensor 1314 coupled to the outer frame 1320 at an elongate portion 1316, and an outflow sensor 1310 coupled to the outer frame 1320 at an elongate portion 1312. Elongate portion 1316 and elongate portion 1312 may function to position inflow sensor 1314 and outflow sensor 1310, respectively, relative to the outer frame 1320 to enable accurate, consistent, and/or repeatable pressure measurements, reduce interference with blood flow through device 1300, and/or reduce areas of stagnate blood flow. Inflow sensor 1314 may represent a pressure sensor, for example, a strain gauge, a capacitive pressure sensor, a piezoelectric sensor, etc. Similarly, outflow sensor 1310 may be a pressure sensor, for example, a strain gauge, a capacitive pressure sensor, a piezoelectric sensor, etc. As such, a pressure differential may be measured across the device 1300. For example, a differential between a pressure measured by the inflow sensor 1314 and an outflow sensor 1310 may be an indication of an amount of modulation the device 1300 is performing. In such an example, a relatively large differential may be indicative of a large restriction within the device 1300, while a relatively small or lack of pressure differential may be indicative of a small restriction in the device 1300 or a lack of restriction in the device 1300. Additionally, or alternatively, a differential between a pressure measured by the inflow sensor 1314 and a pressure measured by the outflow sensor 1310 may be used to determine an amount of flow modulation the device 1300 may perform to reduce pressure in a heart of a subject. For example, the device 1300 may temporarily modulate or decrease flow (or increasing resistance) in order to reduce pressure to the heart.

While the device 1300 is depicted without a skirt membrane, a skirt, such as skirt membrane 122 may be wrapped around an exterior surface of the coated or uncoated frame of device 1300. The skirt membrane 122 may end within a threshold distance of the inflow end 102 of the device 1300. For example, the threshold distance may be about 8 mm to about 12 mm from the inflow perimeter 124; about 8 mm to about 9 mm from the inflow perimeter 124; about 9 mm to about 10 mm from the inflow perimeter 124; about 10 mm to about 11 mm from the inflow perimeter 124; or about 11 mm to about 12 mm from the inflow perimeter 124. A length ls1 of the skirt membrane 122 may be about 15 mm to about 19 mm; about 15 to about 16 mm; about 16 mm to about 17 mm; about 17 mm to about 18 mm; or about 18 mm to about 19 mm.

The skirt membrane 122 may function to reduce blood stasis and/or pooling around the implanted device 1300. For example, the skirt membrane 122 may be at least partially incorporated into an inner wall of a blood vessel to reduce or eliminate clotting and/or blood stasis within cells and struts of the device 1300 by providing an effective seal between the skirt membrane 122 and the blood vessel wall.

In some examples, as shown in FIG. 8, the device 1300 may further include one or more sensor modules 806 (e.g., a sensor 1310 and/or a sensor 1314) for detecting a pressure in the blood vessel, a microprocessor (e.g., processor 808) electrically coupled to the sensor modules 806, and/or a power source (e.g., power source 814) electrically coupled to an actuator (e.g., actuation device 812) associated with device 1300, the microprocessor 808, and/or the sensor modules 806. For example, the sensor modules 806 may sense characteristics of blood flow in the blood vessel (e.g., blood pressure) and may cause the microprocessor 808 to provide signals to the actuator (e.g., actuation device 812). In operation, the microprocessor 808 can receive a signal from the sensor modules 806 that is indicative of a pressure in the blood vessel. The microprocessor 808 can process the signal and generate and provide a control signal (e.g., via control devices 810) to trigger expansion or reduction of expandable member 214 based on the sensed pressure in the blood vessel.

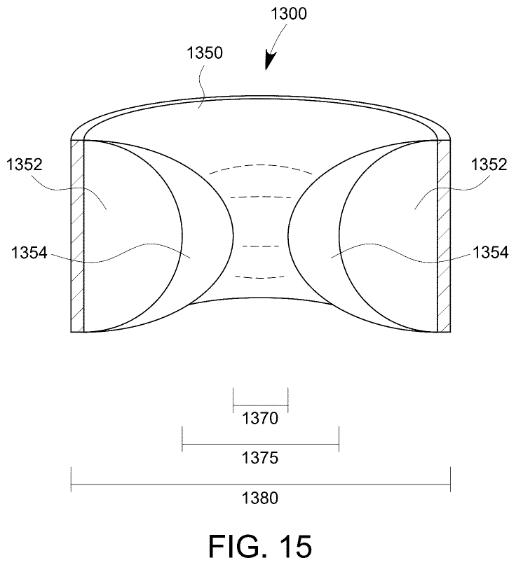

FIG. 13 illustrates a cross-sectional view of the device 1300 for modulating blood flow in a blood vessel. The device 1300 includes an expandable annulus member 1350 that is at least partially coupled to the inner surface 1365 (shown in FIG. 13) defined by the outer frame 1320 (shown in FIG. 12). While a single expandable annulus member 1350 may be utilized in the examples described herein, one or more additional expandable annulus members may be utilized within device 1300, such as is depicted in FIG. 15. Additionally, an annulus member such as annulus member 1350 may be formed from multiple individually members, such as individual wedge-shaped members extending around the inner surface to form a generally annular form.

The expandable annulus member 1350 may be communicatively coupled to an inflation control device 210 by an inflation line 1216. The inflation control device 210 may cause inflation fluid to be transported, shown by arrow 1420, to the expandable annulus member 1350 to expand member 1350 (and/or to expandable annulus sub-members, if present). Similarly, the inflation control device 210 may draw inflation fluid, shown by arrow 1410, from the expandable annulus member 1350 (and/or expandable annulus sub-members) to contract the expandable annulus member 1350.

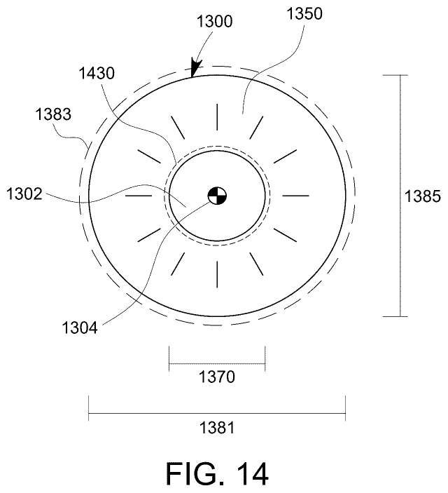

The expandable annulus member 1350 may be formed by profile 1360 that may range from parabolic when at least partially expanded to approximately linear and approximately parallel to an inner surface 1365 of the device 1300. Further, the expandable annulus member 1350 may be three dimensionally defined by the area 1353 between the profile 1360 and the inner surface 1365 of the device 1300, at least partially revolved around a longitudinal axis 1362 of the device 1300. The longitudinal axis 1362 passes through the centroid 1304 (shown in FIG. 14) of the device 1300.

Modulation of blood flow through the device 1300 is controlled by changes in the inflation state of the expandable annulus member 1350. For example, increasing inflation fluid in the expandable annulus member 1350 may cause the expandable annulus member 1350 to expand, increasing the width 1364, or distance, between the parabolic peak 1355 of the profile 1360 and the inner surface 1365 of the device 1300. When the expandable annulus member 1350 is expanded, the flow area 1430 within the orifice 1302 defined by the device 1300 (shown in FIG. 14) is reduced as shown by narrowing passage 1370 of the orifice 1302. As the flow area 1430 of the orifice 1302 defined by the device 1300 is decreased, the flow restriction of the device 1300 is increased. Inversely, decreasing inflation fluid in the expandable annulus member 1350 causes the expandable annulus member 1350 to contract, decreasing the width 1364, or distance, between the parabolic peak 1355 of the profile 1360 and the inner surface 1365 of the device 1300. When the expandable annulus member 1350 is contracted, the flow area 1430 of the orifice 1302 defined by the device 1300 is increased as the passage 1370 of the orifice 1302 defined by the device 1300 is increased. As the flow area 1430 of the orifice 1302 defined by the device 1300 is increased based on inflation of member 1350, the flow restriction of the device 1300 is decreased. In some embodiments, as shown in FIG. 14, the diameter 1381 of a portion of the blood vessel 1383 in which the device 1300 is implanted may be approximately equal to the unrestricted diameter 1380 of the device 1300, such that the cross-sectional area 1385 of the blood vessel 1383 is approximately equal to the cross-sectional area of the device orifice 1302 in an unrestricted state. Area 1385 and area 1430 are both cross-sectional with respect to fluid flow.

In some embodiments, member 1350 may include one or more additional expandable members (not shown, but similar to cavities 1352, 1354 of FIG. 15) within the member 1350 for expansion and deflation based on sensed pressures. The one or more additional expandable members may function to control major or minor increases or decreases in inflation fluid to adjust the flow restriction of the device. For example, a first additional expandable member may be filled to a selected capacity and may maintain the capacity. This may modify flow restriction of the device, but not far enough to induce pressure changes in the vessel in which device 1300 is implanted. At a later time, an increase in pressure may be sensed in the vessel and one or more of the additional expandable members or member 1350 may be triggered to receive inflation fluid to increase in size, thereby alleviating the pressure by further restricting blood flow in the vessel at a juncture near to the member 1350 and/or other additional expandable members. In this way, the combination of devices may be used to alleviate pressure for the patient having device 1300 implanted at a faster rate because the initial additional expandable member can be inflated to a threshold level before a pressure increase is detected. The patient will not have to wait for the two or more devices to be inflated because a first device may be held at the threshold level of inflation.

For embodiments with more than one inflatable chamber, one or more chambers may be pre-inflated (e.g., during device deployment by the user/surgeon etc., such as by using a pump or other inflation element that is in the operating room). The pre-inflated chambers can be scaled and/or in fluid isolation from the active pump of the system (e.g., pump 210). The remaining chamber or chambers can be in active fluid connection with the pump (e.g., pump 210), and are thereby actively controlled in their inflation/deflation. Having the pump drive only a portion of the multiple chambers can thereby reduce the amount of fluid needed to be actively pumped to operate the device.

An example threshold level of inflation may include about 40% to about 80% of full occlusion of the first expandable member; about 40% to about 45%; about 45% to about 50%; about 50% to about 55%; about 55% to about 60%; about 60% to about 65% of full occlusion of the first expandable member; about 65% to about 70% of full occlusion of the first expandable member; about 70% to about 75% of full occlusion of the first expandable member; or about 75% to about 80% of full occlusion of the first expandable member. In some embodiments, the threshold level of inflation may include providing enough inflation fluid in the first expandable member until a baseline flow restriction level is achieved. This baseline may be determined based on detected pressure from one or more sensors 1310, 1314. The initial inflation of the first expandable member may be performed actively or passively. In some embodiments, the initial inflation of the first expandable member may be performed actively or passively during implant of the device.

In some embodiments, such as that shown in FIG. 15, the expandable annulus member 1350 may include an annulus volume defined by an inner cavity 1352 that is expanded and contracted for flow modulation within the blood vessel. The inner cavity 1352 may be actuated from any point between, and including, fully contracted to fully expanded for predefined flow modulation control. Alternatively, inner cavity 1352 may be passively inflated to achieve a predefined flow reduction, for example a minimum flow reduction or a flow reduction above or at a predefined threshold. In some embodiments, the device 1300 may include an annulus volume defined by a first inner cavity 1352 and a second inner cavity 1354, such as where the second inner cavity 1354 is concentrically located within the first inner cavity 1352 as depicted in FIG. 15. The first inner cavity 1352 may be actively or passively inflated to achieve a minimum desired flow reduction, for example a minimum flow reduction or a flow reduction above or at a predefined threshold, followed by passive or active actuation of second inner cavity 1354 to achieve a predefined flow reduction. The active or passive inflation may be based on the differential pressure measurements, such as that from sensors 1310 and/or 1314 of FIG. 12.

In some embodiments, the first inner annulus member 1352 may function to provide a level of flow restriction for device 1300 while the second inner annulus member 1354 provides an incremental additional flow restriction for device 1300. For example, member 1352 may be expanded with inflation fluid to a first predefined fill level (e.g., during implantation of the device into the patient) to provide a static pressure measurement (detected by one or more of sensors). If, over time, an increase in the pressure is detected to be beyond a predefined pressure threshold described elsewhere herein, then the member 1354 may also be expanded with inflation fluid (e.g., from another port) to alleviate the added pressure at the heart (or kidneys). The members 1352, 1354 may function in tandem to provide pressure relief by expanding and/or deflating according to sensed pressures. Note that the operation of the annulus members 1352, 1354 could be reversed, such as where expandable annulus member 1354 is filled to a predefined fill level, and the expandable annulus member 1352 is selectively expanded with inflation fluid according to sensed pressures.

Depending on the particular embodiment, chamber 1352 may be pre-filled (e.g., during delivery/deployment) to a pre-set level, with only chamber 1354 being actively inflated/deflated via the pump/control 210. Alternatively, chamber 1354 could be pre-inflated, with only chamber 1352 actively inflated/deflated via pump control 210. Or both 1352 and 1354 could be actively inflated/deflated via pump control 210, which could be accomplished via separate fill lines for independent control of the inflation/deflation of each chamber, and/or separate pumps, etc.

Embodiments with multiple cavities defining the annulus volume may be advantageous in modulation control. For example, the first inner cavity 1352 may be fluidly coupled to a first inflation line (e.g., inflation line 1216). The second inner cavity 1354 may be fluidly coupled to a second inflation line (not shown). Alternatively, the second inner cavity 1354 may be fluidly coupled to a second lumen (not shown) of an inflation line in which a first lumen is connected to the first inner cavity 1352. Being fluidly coupled to either a second inflation line, or a second lumen of an inflation line, allows the separate and independent expansion and contraction of the first inner cavity 1352 and the second inner cavity 1354. As such, the first inner cavity 1352 may be expanded prior to the second inner cavity 1354. When a modulation of the blood vessel is triggered (e.g., a pressure greater than a predefined pressure), some device 1300 embodiments may expand the first inner cavity 1352, thus achieving a first restriction value provided by the expansion of the first inner cavity 1352. The first restriction value may be large enough that flow modulation is considerable. For example, the first inner cavity 1352, when partially or fully expanded, may reduce the flow area 1430 of the device orifice 1302 (shown in FIG. 14) from about 100% (i.e., area 1430 of the unrestricted device orifice 1302) to a flow area 1430 of about 20% to about 60%; about 20% to about 40%; about 40% to about 60%; about 30% to about 50%; about 30% to about 60%; about 20% to about 50%; about 25% to about 45%; etc. Said another way, the first inner cavity 1352, when partially or fully expanded, may consume, block, or obstruct about 40% to about 80%; about 60% to about 80%; about 40% to about 60%; about 50% to about 70%; about 40% to about 70%; about 50% to about 80%; about 55% to about 75%; etc. of the device orifice 1302.

In some embodiments, after the expansion of the first inner cavity 1352, the second inner cavity 1354 may be expanded to further reduce the flow area 1430 of the device orifice 1302. For example, the second inner cavity 1354, when partially or fully expanded, may further reduce the flow area 1430 of the device orifice 1302 (shown in FIG. 14) to a flow area 1430 of about 5% to about 50%; about 5% to about 20%; about 20% to about 40%; about 30% to about 50%; about 5% to about 15%; about 10% to about 30%; about 10% to about 40%; about 10% to about 20%; about 20% to about 0%; etc. of the unrestricted flow area 1430 of the device orifice 1302 (i.e., the area 1385 of the blood vessel). Said another way, when the first inner cavity 1352 and the second inner cavity 1354 are partially or fully expanded, the first inner cavity 1352 and the second inner cavity 1354 may together consume, block, or obstruct about 50% to about 90%; about 80% to about 95%; about 60% to about 80%; about 50% to about 70%; about 85% to about 95%; about 70% to about 90%; about 60% to about 90%; about 80% to about 90%; about 80% to about 100%; etc. of the device orifice 1302. The second inner cavity 1354 may be actuated from any point between, and including, fully contracted to fully expanded for predefined flow modulation control.

In some embodiments, the first inner cavity 1352 may be maintained in an expanded or fully expanded state, and an expansion status of the second inner cavity 1354 may be modulated based on one or more pressure measurements received from the one or more sensors 1310, 1314. For example, the first inner cavity 1352 may be maintained in an expanded state such that the flow area 1430 of the device orifice 1302 may be reduced to a flow area 1430 of about 20% to about 60%; about 20% to about 40%; about 40% to about 60%; about 30% to about 50%; about 30% to about 60%; about 20% to about 50% about 25% to about 45%; etc. The second inner cavity 1354 may be further modulated based on one or more sensed parameters from the one or more sensors 1310, 1314 to further reduce the flow area 1430 of the device orifice 1302 to a flow area 1430 of about 5% to about 50%; about 5% to about 20%; about 20% to about 40%; about 30% to about 50%; about 5% to about 15%; about 10% to about 30%; about 10% to about 40%; about 10% to about 20%; about 0% to about 30%; etc. Said another way, the second inner cavity 1354 may be further modulated based on one or more sensed parameters from the one or more sensors 1310, 1314 to further block or obstruct, in collaboration with the first inner cavity 1352, about 50% to about 95%; about 80% to about 95%; about 60% to about 80%; about 50% to about 70%; about 85% to about 95%; about 70% to about 90%; about 60% to about 90%; about 80% to about 90%; about 70% to about 100%; etc. of the device orifice 1302.

In embodiments such as FIG. 15 with multiple chambers (1352, 1354), one or more chambers may be pre-inflated (e.g., during device deployment by the user/surgeon etc., such as by using a pump or other inflation element that is in the operating room). For example, one cavity (e.g., cavity 1352) could be pre-inflated to a desired level (e.g., desired occlusion percentage) during initial deployment, with another cavity (e.g., cavity 1354) having its volume actively controlled via pump system 210 based on blood pressure sensor data, etc.

Embodiments of devices for modulating blood flow in a blood vessel which include a first inner cavity 1352 and a second inner cavity 1354 may exploit the physical nature of incompressible fluid flow through a restriction. Observing Bernoulli's Principle/equation, provides flow relationships to pressure drop (ΔP):

$$Δ P = ( 1 / 2 ) ρ ( {V}_{2}^{2} - {V}_{1}^{2} )$$

Where p is the density of an incompressible fluid, V₁ is the velocity of the fluid prior to the restriction, and V₂ is the velocity of the fluid at the restriction. Observing the Continuity Equation, relates the unrestricted velocity (V₁) prior to the restriction and velocity (V₂) at the restriction:

$${V}_{2} = {V}_{1} ( {A}_{1} / {A}_{2} )$$

Where A₁ is the cross-sectional area of the lumen (i.e., a blood vessel in which the device 1300 is implanted) and A₂ is the cross-sectional area of a restriction (i.e., the flow area 1430 of the device orifice 1302) in a lumen. As the restriction (A₂) is reduced from 100% of A₁ (i.e., area 1430 of the unrestricted device orifice 1302) to 0% of A₁, V₂ parabolically increases toward a vertical asymptote at 0%. For example, reduction of area (A₂) at the restriction to a range of 95% to 55% of A₁ (i.e., flow area 1430 of the unrestricted device orifice 1302) would result in an increase of velocity (e.g., (V₂/V₁)*100%) of approximately 5% to approximately 80%, respectively. In contrast, reduction of area (A₂) at the restriction to a range of 50% to 10% of A₁ (i.e., area 1430 of the unrestricted device orifice 1302) would result in an increase of velocity (e.g., (V₂/V₁)*100%) of approximately 200% to approximately 1000%, respectively. Pressure drop (ΔP) is dependent on the square of this velocity change. As such, effects of a restriction on flow are relatively small until the restriction becomes large because the velocity change is not significant for small area reductions. In light of the principles above, embodiments of devices for modulating blood flow in a blood vessel with a first inner cavity 1352 and a second inner cavity 1354 may be advantageous in their actuation. For example, when actuating, a flow modulating device 1300 may rapidly expand the first inner cavity 1352 (or maintain the first inner cavity 1352 in an expanded state), such that the flow area 1430 of the device orifice 1302 is considerably smaller than the area 1385 of the blood vessel 1383, as shown in FIG. 14. The second inner cavity 1354 may be used to further control flow modulation through the device 1300 by controlling further reduction of the area 1430 of the device orifice 1302 within a range of considerable pressure drop (ΔP). Said another way, and as shown in FIG. 15, the inner cavity 1352 may be expanded rapidly, or maintained in an expanded state, to reduce the device orifice 1302 to a diameter 1375, and the second inner cavity 1354 may be expanded to reduce the device orifice 1302 to the diameter 1370. Further, the device 1300 may be advantageous in modulation control due to the reduced volumetric control of the second inner cavity 1354.

A flow modulating device 1300 as shown in FIGS. 12-15 may be advantageous in that the smooth, venturi-like shape of the expandable annulus member 1350 of the device 1300 does not provide refuge for eddy currents or stagnant fluid pockets. As such, the smooth, venturi-like shape of the expandable annulus members 1350 may discourage pooling/stagnation of blood flow.

In some examples, the one or more expandable annulus members 1350, 1352, 1354 can include a compliant material. In some examples, the one or more expandable annulus members 1350, 1352, 1354 can be formed of a compliant material. In some examples, the one or more expandable annulus members 1350, 1352, 1354 can consist essentially of a compliant material. A compliant material may exhibit a burst pressure of about 0 atmospheres (atm) to about 2 atm. In some examples, a compliant material may be able to expand about 20 percent to about 900 percent. Non-limiting examples of compliant materials include silicones, latex, polyvinyl chloride, polyolefin copolymer, or a combination thereof. The one or more expandable annulus members 1350, 1352, 1354 may be formed of a compliant material to provide an advantage of maintaining a smooth surface in all configurations of expansion. Such a compliant material may reduce or prohibit deformations in the material in which blood may be trapped.

In some instances, the one or more expandable annulus members 1350, 1352, 1354 can include a semi-compliant material. In some examples, the one or more expandable annulus members 1350, 1352, 1354 can be formed of a semi-compliant material. In some examples, the one or more expandable annulus members 1350, 1352, 1354 can consist essentially of a semi-compliant material. A semi-compliant material may exhibit a burst pressure of about 1 atm to about 25.5 atm. In some examples, a semi-compliant material may be able to expand about 10 percent to about 20 percent. Non-limiting examples of semi-compliant materials include polyethylene terephthalate, nylons, thermoplastic polyurethanes, thermoplastic elastomers, or a combination thereof.

In some instances, the one or more expandable annulus members 1350, 1352, 1354 can include a non-compliant material. In some examples, the one or more expandable annulus members 1350, 1352, 1354 can be formed of a non-compliant material. In some examples, the one or more expandable annulus members 1350, 1352, 1354 can consist essentially of a non-compliant material. A non-compliant material may exhibit a burst pressure of about 1 atm to about 25.5 atm. In some examples, a non-compliant material may be able to expand about 0 percent to about 10 percent. Non-limiting examples of non-compliant materials include polyethylene terephthalate and like materials.

In some examples, one or more of the expandable annulus members 1350, 1352, 1354 include a compliant material, and one or more of the expandable annulus members 1350, 1352, 1354 include a non-compliant material. Expandable members having different properties or including different materials may, for example, achieve different filling rates of the expandable members, make a subset of the one or more expandable members more resistant to bursting, achieve various degrees of expansion of the one or more expandable members, allow differential filling of a blood vessel or a cross-sectional area of a lumen of the frame 1320 based on a material of the one or more expandable materials, and the like. In some examples, one or more of the expandable annulus members 1350, 1352, 1354 may include a blend of a compliant material and a semi-compliant material. In some examples, one or more of the expandable annulus members 1350, 1352, 1354 may include a blend of a non-compliant material and a semi-compliant material. In some examples, one or more of the expandable annulus members 1350, 1352, 1354 may include a blend of a compliant material and a non-compliant material.

### Methods

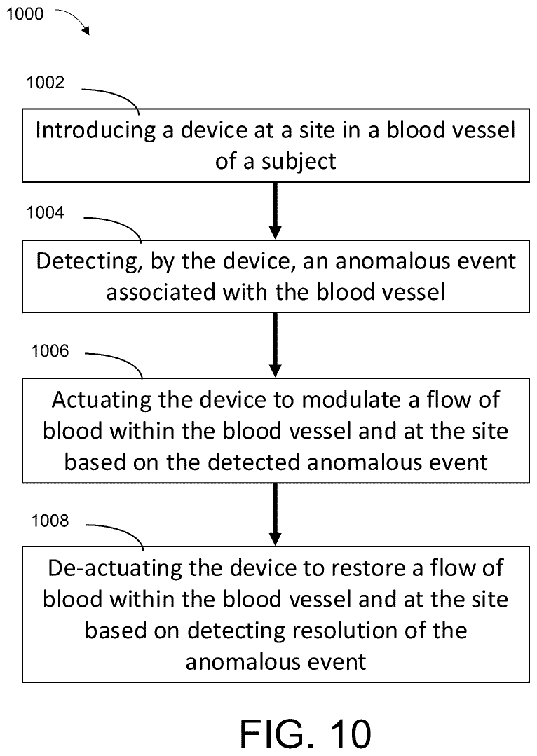

FIG. 10 is a flow diagram of an example process 1000 of modulating blood flow through one or more blood vessels. The process 1000 functions to reduce cardiac blood flow at a target site in a blood vessel of a subject. In some embodiments, the process 1000 functions to modulate a volume of blood flowing from the blood vessel into a right atrium to decrease right atrial pressure. The process 1000 may be used for blood flow regulation in a target site within the vena cava, the SVC, or the IVC, but can additionally, or alternatively, be used for any suitable applications, clinical or otherwise. In general, process 1000 may be used with any of the devices described herein. In some examples, the process 1000 may be a method of treatment for reducing cardiac blood flow at a target site in a blood vessel of a subject.

As an example, the device used with process 1000 may include device 100, device 150, device 200, and device 1300. For example, the device 100, 200, or 1300 may include a frame 106, 1320 having an inflow end 102, an outflow end 104, and a lumen therethrough. In some examples, the device 100 does not include a frame, such as in the example of device 150. The device 100 includes the expandable member 114, 1350 coupled to the inflation line 116, 1216. The expandable member 114, 1350 defines a volume for receiving an inflation fluid therein.

In some examples, the process 1000 may be a method of treatment for modulating blood flow in an SVC in a subject having chronic kidney disease and/or chronic heart failure. The method of treatment may include introducing a vessel occlusion device at a site in a blood vessel of a subject (block 1002). For example, the devices described herein may be partially or fully housed by a frame (e.g., a stent). The outer frame 106, 206, 1320 of the device 100, 200, 1300, for example, may be introduced to a vessel or tissue site using a delivery system. The outer frame 106, 206, 1320 may be self-expanding (i.e., released during delivery) or may be expanded during delivery (e.g., via an expandable balloon). In a coronary procedure, a catheter tip and/or catheter may be configured to pass from the right atrium into the coronary sinus to implant the device 100, 200, or 1300. For access to the venous circulation, for example, a catheter tip and/or catheter may be configured to pass from the radial artery into the SVC to implant the device 100, 200, or 1300 into a portion of the SVC. Further, for central venous access, a catheter tip and/or catheter may be passed from the femoral vein into the IVC to implant the device 100, 200, or 1300 into a portion of the IVC.

At block 1004, the method of treatment may include detecting, by the device 100, 200, 1300, an anomalous event (or several events) associated with the blood vessel. For example, the process 1000 may include detecting an increasing blood pressure in the right atrium of the subject. In particular, the device 100, 200, 1300 may include one or more sensor modules (e.g., module 117, sensor 1310, sensor 1314, and/or module 156, safety sensors, pressure sensors, processors, etc.) to detect the increased blood pressure and/or the rate of increase. In some examples, the one or more sensors may be programmed to detect one or more predefined blood pressure threshold levels where detecting blood pressure above the predefined blood pressure threshold triggers the device to actuate the expandable member 114, 1350 and lower the blood pressure. In some examples, actuating the device 100, 200, 1300 may cause a partial occlusion of blood flow through the blood vessel in which device 100, 200, 1300 is implanted.

In some examples, the one or more predefined thresholds may pertain to a rate of pressure increase. In such examples, the one or more sensors may detect that the rate is above the predefined threshold level for rate increase and may cause the device 100, 200, 1300 to actuate to modulate a flow of blood within the blood vessel by triggering flow of fluid into one or more expandable members 114, into one or more expandable annulus members 1350, and/or into one or more inner cavities of the expandable annulus member 1350, as shown at block 1006. In some examples, the device 100, 200, 1300 may be actuated based on a sensed anomalous event (or several events). Actuating the device 100, 200, 1300 may trigger modulation of a flow of blood within the blood vessel at the device 100, 200, 1300 site based on the detected anomalous event. For example, actuating the device 100, 200, 1300 may cause a partial or other occlusion of the blood vessel at the site of the device. For example, if the device 100, 200, 1300 is implanted into the SVC of a subject having chronic kidney disease and/or chronic heart failure, the device 100, 200, 1300 can be actuated to increase the amount of fluid in one or more inner cavities of the expandable annulus member 1350 or the amount of fluid in one or more expandable members 114 to modulate a volume of blood flowing from the blood vessel into a right atrium so as to decrease right atrial pressure. With reference to FIG. 15, actuation of the device 1300 may be partial expansion of the first inner cavity 1352, may be full expansion of the first inner cavity 1352 and partial expansion of the second inner cavity 1354, or may be full expansion of the first inner cavity 1352 and full expansion of the second inner cavity 1354.

In some examples, the one or more sensors 1310 and/or 1314 and/or sensor modules 117, 156 may detect pressure in the blood vessel in which device 100 is implanted to cause a selective increase or a selective decrease of fluid in the expandable member 114 or expandable annulus members 1350 based on the detected pressure sensed by the sensor modules 117, 156 or sensor 1310 and/or 1314, respectively. In some examples, at least one of the sensor modules 117, 156 may be coupled to a distal end (e.g., distal end 216b in FIG. 2B) of the inflation line 116 and to measure or sense right atrial pressure.

In some examples, the device 100, 200, 1300 include a first pressure sensor 117, 1314 arranged to detect right atrial pressure and a second pressure sensor 156, 1310 arranged to detect SVC pressure or renal pressure. The device 100, 200, 1300 may be programmed to perform a calibration process on the expandable member 114 or expandable annulus member 1350. For example, a calibration process may include defining a pressure threshold gradient for the subject, determining a difference in pressure between the first pressure sensor module 117, 1314 and the second pressure sensor 156, 1310, and automatically calibrating an amount of fluid to be injected into or removed from the expandable member 114 or expandable annulus member 1350 based on the determined difference in pressure when the determined difference in pressure exceeds the pressure threshold gradient.

Further, one or more sensors/sensor modules may be used in conjunction with any of the devices and systems herein to measure one or more physical characteristics of a subject having one of the devices described herein implanted. For example, it may be beneficial to measure whether the subject is standing, sitting, or laying. In addition, the pressure thresholds for activating the device may be influenced by the activity of the subject. For example, it may be beneficial to realize the subject is exercising, as this would elevate pressures and may cause an adjustment in pressure thresholds. Characteristics described above may be measured by a pressure sensor in blood vessels of other portions of the body, a gyroscopic sensor for changes in angular position, an accelerometer for changes in acceleration, a heart rate sensor, a sensor measuring a size of a blood vessel, or any other sensors for measuring physical characteristics. The described characteristics, individually or in combination, may be received by a microprocessor and processed to cause changes in valve position (using an actuating device) based on the sensed characteristics.

At block 1008, the process 1000 for the method of treatment may include de-actuating the device 100, 200, 1300 to restore a flow of blood within the blood vessel and at the site based on detecting resolution of the anomalous event. For example, when one or more sensors/sensor modules of device 100, 200, 1300 detects a resolution or a change of the blood pressure (e.g., the blood pressure is below the threshold level), then the device 100, 200, 1300 may trigger a de-actuation of blood flow modulation, which may function to decrease a level of fluid in one or more expandable members 114 or expandable annulus member 1350 to maintain or regain a flow of blood within the blood vessel. Maintaining a flow of blood within the blood vessel may include ensuring the device 100, 200, 1300 is held in a particular state of blood regulation such that one or more components of the device may be held stationary over time. Regaining a flow of blood within the blood vessel may include relaxing any blood occlusion components or structures (e.g., expandable member 114, expandable annulus member 1350) such that the flow of blood may pass through the blood vessel unencumbered. For example, the device 100, 200, 1300 may be configured to function in an unrestricted blood flow state or a restricted blood flow state. The unrestricted blood flow state (e.g., collapsed configuration) corresponds to the expandable member collapsing to allow blood flow through the blood vessel. The restricted blood flow state (e.g., expanded configuration) corresponds to the expandable member expanding to reduce blood flow through the blood vessel.

In some examples, the device 100, 200, 1300 may further includes an internal counter to count time between inflation and/or deflation cycles. The process 1000 may further include using the internal counter to track and count a time between inflation cycles for the expandable member 114 or expandable annulus member 1350 and impose a time constraint between inflation cycles of the device 1300. The process 1000 may further include using the internal counter to track and count a time between deflation cycles for the expandable member 114 or expandable annulus member 1350 and impose a time constraint between inflation cycles of the device 100, 200, 1300.

### Example Implantation of Flow Modulating Devices

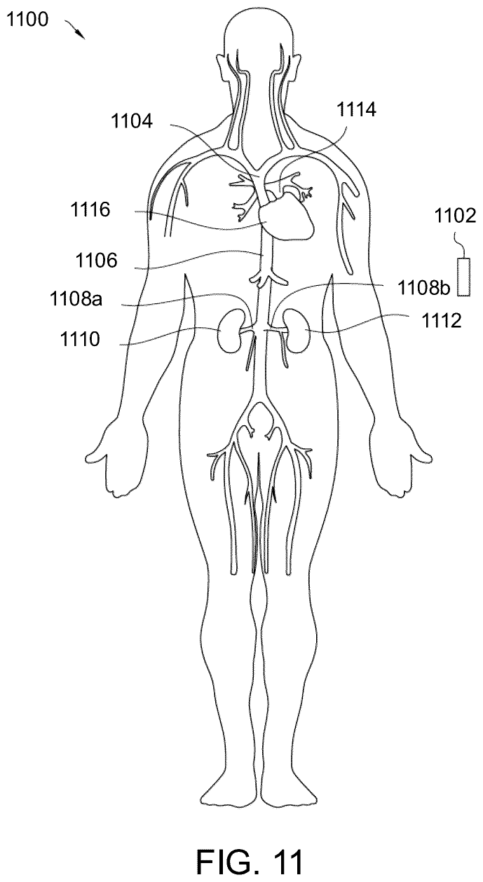

FIG. 11 illustrates a schematic representation of portions of a subject 1100. The flow modulating devices described herein (represented in FIG. 11 by device 1102) may be introduced (e.g., implanted) in vasculature of the body. In general, the device 1102 may represent any of the flow modulating devices described herein (e.g., device 100, 200, 1300) and may include the same or similar functionality and/or structures. In some examples, the device 1102 may be implanted in or near to a portion of the SVC 1104. In some examples, the device 1102 may be implanted in or near to a portion of the IVC 1106. The subject 1100 is illustrated with a representation of a portion of the vasculature system to generally illustrate the SVC 1104 and the IVC 1106 within the subject 1100. However, it is to be understood that no dimensions or relative sizes of components may be inferred from the relative sizes and dimensions of elements in the figures.

The subject 1100 includes a number of vessels and organs that may circulate blood throughout the body. For example, renal veins 1108a and 1108b drain blood from respective right kidney 1110 and left kidney 1112. Renal veins 1108a and 1108b connect to the IVC 1106. Blood from the aorta 1114 flows to the IVC 1106. Blood travels from the aorta 1114 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1106 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized. For example, because blood flow from the splanchnic venous circulation is directed through hepatic veins and into the IVC 1106, devices (as described herein) may be placed into the IVC 1106 to limit blood flow to allow the splanchnic venous circulation to expand with increased blood volume. This may also allow the body to accumulate blood volume in the splanchnic venous circulation, which can maximize the downstream drop of pressure relative to upstream increase of pressure. Similarly, devices (as described herein) may be placed into the SVC 1108 to limit blood flow to allow the reservoir to expand with increased blood volume. Furthermore, the flow modulating devices described herein may be placed in either the IVC 1106 and/or SVC 1108 to alleviate pressure in the right side of the atrium of the heart 1116 and/or regulate renal venous pressure and kidney function. Another example positioning of a flow modulating device may be in the IVC below the renal veins. This positioning may have a similar effect as the SVC location, as it may allow the flow modulating device to maintain renal venous pressure, which can correlate with sustained renal function and diuresis.

In some examples, the flow modulating device 1102 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1102 may be used as a method of treatment to regulate pressure in the right atrium of the heart. Further, the flow modulating device 1102 may be used as a method of treatment to improve function of the kidneys in patients having reduced kidney function due to pressure in the venous system.

For example, any of the implantable devices and/or systems described herein may be configured to modulate a volume of blood flowing from the SVC into a right atrium to decrease right atrial pressure.

Further for example, any of the implantable devices and/or systems described herein may be used to perform a method including restricting blood flow within a blood vessel. Still further for example, any of the implantable devices and/or systems described herein may be used to perform a method of treatment for a subject having one or both of: congestive heart failure or chronic kidney disease. The method may include restricting blood flow within the blood vessel.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve-based system (e.g., with a manifold), hydraulics, pneumatics, optical actuators, thermal actuators, and/or other actuator using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include an actuator (e.g., a linear actuator) coupled to a control element of the flow management device. The linear actuator tensions the control element to position the valve of the flow management device in a restricted blood flow state. Alternatively, the linear actuator releases tension in the control element to position a valve, a membrane, or other material in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, decreasing a temperature of a thermal sensitive fluid (e.g., via a heat source, changes in body temperature, etc.) causes the piston to compress the fluid to tension the control element to move the valve into the restricted blood flow state. Alternatively, increasing the temperature of the thermal sensitive fluid causes the piston to decompress the fluid to release tension in the control element to move the valve to the unrestricted blood flow state.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, actuation members, tubes, etc.), anatomy responses (e.g., changes in vessel inner diameter, intra-vessel pressure, etc.), blood movement, or the like.

Any of the implantable or flow modulating devices described herein may be coated with a polymer (e.g., silicones, poly (urethanes), poly (acrylates), or copolymers such as poly (ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly(ethylene terephthalate), while the nonwoven fabrics may be made from expanded poly (tetrafluoroethylene). Some textiles may also or alternatively include silk or silk-based materials.

Further, any of the pull wires, sutures, frames/stents, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent (e.g., a frame and/or a braid). The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

As used herein, examples referring to devices that allow blood to flow through the blood vessel may represent the blocking or unblocking of blood flow through a lumen of the particular device (rather than through the vessel itself) when the device is implanted. For example, portions of the blood vessel may be implanted with one or more of the devices described herein and those portions may be allowed or disallowed to flow blood through the lumen of the device within the vessel resulting in partially blocking or unblocking flow through the portions of the blood vessel implanted with such a device.

The stents described herein may be configured to house all or a portion of the flow modulating devices described herein. Such stents may include an assembly with struts (e.g., strut members) members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may represent an outer frame. The outer frame may have a form and structure that varies. For example, the struts and/or joints may form a mesh-like structure. The struts may be interconnected in such a way as to form a shaped pattern of cells. For example, any number of struts may form a ring of the stent (e.g., frame) such that the struts are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of struts interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the SVC. Further, for central venous access, a catheter tip and/or catheter may be configured to pass from the femoral vein into the IVC.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., SVC, IVC, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, SVC, or IVC, including for example delivery catheters, cannulas, and/or trocars. It will be understood that other types of medical implant devices and/or procedures can be delivered to the coronary sinus, SVC, IVC, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the control device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

### EXAMPLES

Example 1. A device for modulating blood flow in a blood vessel, the device comprising: an expandable member comprising an inflation port coupled to an inflation line, the expandable member defining a volume configured to receive an inflation fluid therein through the inflation port; and an anchor having a first surface configured to couple to the inflation line along a portion of length of the inflation line and a second surface configured to couple to a wall of the blood vessel.

Example 2. The device of any one of the preceding examples, but particularly Example 1, further comprising at least one pressure sensor module coupled to a distal end of the expandable member and configured to: detect pressure in the blood vessel, with the pressure data used by the device to cause, based on the detected pressure, an increase or a decrease of the inflation fluid in the expandable member.

Example 3. The device of any one of the preceding examples, but particularly Example 2, wherein the at least one pressure sensor module is positioned on the inflation line upstream from the inflation port.

Example 4. The device of any one of the preceding examples, but particularly Example 2, further comprising a safety sensor module configured to interrupt or stop at least one cycle of the modulating of the blood flow by the device in response to detecting a blood vessel pressure above a predefined safety threshold.

Example 5. The device of any one of the preceding examples, but particularly Example 1, wherein the expandable member comprises an inner cavity in fluid communication with the inflation line.

Example 6. The device of any one of the preceding examples, but particularly Example 1, wherein: the inflation line is coupled to a subcutaneously implanted reservoir and pump; and actuation of the pump causes inflation fluid from the reservoir to be pumped into the expandable member to cause partial or full inflation of the expandable member.

Example 7. The device of any one of the preceding examples, but particularly Example 6, further comprising a power source coupled to the pump, the power source including a battery.

Example 8. The device of any one of the preceding examples, but particularly Example 1, wherein the blood vessel comprises a vena cava, a superior vena cava, or an inferior vena cava.

Example 9. The device of any one of the preceding examples, but particularly Example 1, wherein: the anchor is a sleeve configured to receive the inflation line therethrough; the first surface defines an inner surface of the sleeve; and the second surface defines an outer surface of the sleeve.

Example 10. The device of any one of the preceding examples, but particularly Example 1, wherein the inflation line is flexible to enable the expandable member to move (e.g., oscillate) within the blood vessel in response to blood flow through the blood vessel.

Example 11. The device of any one of the preceding examples, but particularly Example 1, wherein the expandable member is adjustable to a plurality of positions between expanded and collapsed, the plurality of positions including at least: an expanded configuration configured to fully occlude the blood vessel; a partially expanded configuration configured to partially occlude the blood vessel; and a collapsed configuration configured to not substantially occlude the blood vessel.

Example 12. The device of any one of the preceding examples, but particularly Example 1, wherein the expandable member is: a substantially spherical shape with a substantially annular cross section when in an expanded position; and configured to inflate up to a maximum elongation of about 900 percent.

Example 13. The device of any one of the preceding examples, but particularly Example 1, wherein the expandable member is: a substantially biconical shape with two substantially congruent cones joined at respective bases when in an expanded position; and configured to inflate up to a maximum elongation of about 300 percent.

Example 14. The device of any one of the preceding examples, but particularly Example 1, wherein the expandable member is coated with thromboresistant material.

Example 15. The device of any one of the preceding examples, but particularly Example 1, wherein the expandable member is coated with a Perylene micro-coating.

Example 16. The device of any one of the preceding examples, but particularly Example 1, wherein the expandable member comprises an inflatable balloon.

Example 17. A flow restrictor for a blood vessel, comprising: an outer frame; an expandable member comprising an inflation port coupled to a first portion of an inflation line, the expandable member defining a volume configured to receive an inflation fluid therein through the inflation port; and an anchor configured to couple a second portion of the inflation line to an internal surface of the outer frame to allow the expandable member to oscillate or otherwise move within the outer frame or within a portion of the blood vessel in response to blood flow through the blood vessel.

Example 18. The flow restrictor of any one of the preceding examples, but particularly Example 17, wherein: the first portion of the inflation line is within a first threshold distance of a distal end of the inflation line; and the second portion of the inflation line is within a second threshold distance of a proximal end of the outer frame.

Example 19. The flow restrictor of any one of the preceding examples, but particularly Example 17, further comprising: at least one pressure sensor module configured to detect pressure in the blood vessel, with the detected pressure used by the device to cause an increase or a decrease of inflation fluid in the expandable member based on the detected pressure.

Example 20. The flow restrictor of any one of the preceding examples, but particularly Example 19, wherein the at least one pressure sensor module is coupled to a distal end of the inflation line and configured to measure or sense right atrial pressure.

Example 21. The flow restrictor of any one of the preceding examples, but particularly Example 17, further comprising: at least one pressure sensor module configured to detect pressure in the blood vessel, with detected pressure values used by the device to cause an increase or a decrease of inflation fluid in the expandable member based on the detected pressure, wherein the at least one pressure sensor module is coupled to the inflation line, arranged within a threshold distance of a proximal end of the outer frame, and configured to detect superior vena cava pressure or renal pressure.

Example 22. The flow restrictor of any one of the preceding examples, but particularly Example 21, further comprising a safety sensor module configured to cause an interruption or stoppage of at least one cycle of the flow restricting in response to detecting a blood vessel pressure above a predefined safety threshold.

Example 23. The flow restrictor of any one of the preceding examples, but particularly Example 17, wherein the expandable member is adjustable to a plurality of positions between expanded and collapsed, the plurality of positions including at least: an expanded configuration configured to fully occlude the blood vessel; a partially expanded configuration configured to partially occlude the blood vessel; and a collapsed configuration configured to not substantially occlude the blood vessel.

Example 24. The flow restrictor of any one of the preceding examples, but particularly Example 17, wherein the expandable member is: a substantially spherical shape with a substantially annular cross section when in an expanded position; and configured to inflate up to a maximum elongation of about 900 percent.

Example 25. The flow restrictor of any one of the preceding examples, but particularly Example 17, wherein the expandable member is: a substantially biconical shape with two substantially congruent cones joined at respective bases; and configured to inflate up to a maximum elongation of about 300 percent.

Example 26. The flow restrictor of any one of the preceding examples, but particularly Example 17, wherein the expandable member is coated with thromboresistant material.

Example 27. The flow restrictor of any one of the preceding examples, but particularly Example 17, wherein the expandable member is coated with a Perylene micro-coating.

Example 28. The flow restrictor of any one of the preceding examples, but particularly Example 17, wherein the outer frame comprises an inner surface coated with thromboresistant material.

Example 29. The flow restrictor of any one of the preceding examples, but particularly Example 17, wherein the outer frame comprises: an inflow end, an outflow end, and a lumen defined therethrough, wherein the lumen is radially narrowed along an intermediate portion of the outer frame located between the inflow end and the outflow end, the intermediate portion being configured to constrain the expandable member.

Example 30. The flow restrictor of any one of the preceding examples, but particularly Example 29, wherein the narrowed lumen enables the expandable member to be selected for a maximum expansion defined by a diameter of the narrowed lumen.

Example 31. The flow restrictor of any one of the preceding examples, but particularly Example 17, wherein the outer frame comprises: an inner wall with a first polymeric covering: an outer wall with a second polymeric covering, wherein the first polymeric covering is coupled to portions of the second polymeric covering.

Example 32. The flow restrictor of any one of the preceding examples, but particularly Example 31, wherein the outer frame further comprises: a skirt membrane wrapped around an exterior surface of the second polymeric covering and disposed offset from a lateral centerline at a midpoint of the frame and toward an inflow end of the outer frame.

Example 33. The flow restrictor of any one of the preceding examples, but particularly Example 17, wherein: the blood vessel is a superior vena cava; and the outer frame is configured to be implanted in a vessel branching from the superior vena cava; and the expandable member is tethered to the outer frame at a length such that the expandable member extends beyond an end perimeter of the outer frame and into the superior vena cava substantially adjacent to an azygous junction.

Example 34. The flow restrictor of any one of the preceding examples, but particularly Example 33, wherein: the azygous junction is substantially blocked when the expandable member is in an expanded configuration; and the azygous junction is substantially unblocked when the expandable member is in a collapsed or deflated configuration.

Example 35. The flow restrictor of any one of the preceding examples, but particularly Example 33, wherein the vessel branching from the superior vena cava is a left brachiocephalic vein.

Example 36. The flow restrictor of any one of the preceding examples, but particularly Example 17, wherein: the inflation line is coupled to a subcutaneously implanted reservoir and pump; and actuation of the pump causes inflation fluid from the reservoir to be pumped into the expandable member to cause partial or full inflation of the expandable member.

Example 37. The flow restrictor of any one of the preceding examples, but particularly Example 36, further comprising a power source coupled to the pump, the power source comprising a battery.

Example 38. The flow restrictor of any one of the preceding examples, but particularly Example 17, wherein the blood vessel comprises a vena cava, a superior vena cava, or an inferior vena cava.

Example 39. The flow restrictor of any one of the preceding examples, but particularly Example 17, wherein the expandable member comprises an inflatable balloon.

Example 40. A method of treatment for reducing cardiac blood flow at a target site in a blood vessel of a heart of a subject, the method comprising: introducing a device in the blood vessel, the device comprising: an outer frame; an expandable member comprising an inflation port coupled to a first portion of an inflation line, the expandable member defining a volume configured to receive an inflation fluid therein through the inflation port; and an anchor configured to couple a second portion of the inflation line to an internal surface of the outer frame to allow the expandable member to move (e.g., oscillate), when coupled to the inflation line by the anchor, within the outer frame or blood vessel in response to blood flow through the blood vessel; and actuating the device to modulate a flow of blood within the blood vessel.

Example 41. The method of treatment of any one of the preceding examples, but particularly Example 40, further comprising: de-actuating the device to maintain or regain the flow of blood through the blood vessel.

Example 42. The method of treatment of any one of the preceding examples, but particularly Example 40, wherein the target site includes a portion of a superior vena cava (SVC) of a subject in which the device is implanted, or a portion of an inferior vena cava (IVC) of the subject in which the device is implanted.

Example 43. The method of treatment of any one of the preceding examples, but particularly Example 40, wherein actuating the device causes a partial occlusion of blood flow through the blood vessel.

Example 44. The method of treatment of any one of the preceding examples, but particularly Example 40, wherein the blood vessel is a superior vena cava and the device is configured to be implanted in a portion of the superior vena cava of a subject having chronic kidney disease and chronic heart failure; and the method further comprises modulating a volume of blood flowing from the blood vessel into a right atrium to decrease right atrial pressure.

Example 45. The method of treatment of any one of the preceding examples, but particularly Example 40, further comprising: at least one pressure sensor module configured to detect pressure in the blood vessel, with the device using the detected pressure, via a processor and/or pump and/or valve, to cause a selective increase or a selective decrease of fluid in the expandable member based on the detected pressure.

Example 46. The method of treatment of any one of the preceding examples, but particularly Example 45, wherein the at least one pressure sensor module is coupled to a distal end of the inflation line and configured to measure or sense right atrial pressure.

Example 47. The method of treatment of any one of the preceding examples, but particularly Example 40, wherein the device further includes an internal counter and the method further comprises counting a time between inflation cycles for the expandable member and imposing a minimum or a maximum time constraint between inflation cycles of the device.

Example 48. The method of treatment of any one of the preceding examples, but particularly Example 40, further comprising: a first pressure sensor module arranged to detect right atrial pressure; a second pressure sensor module arranged to detect superior vena cava pressure or renal pressure; wherein the device is further configured to perform a calibration process on the expandable member, the process comprising: defining a pressure threshold gradient for the subject; determining a difference in pressure between the first pressure sensor module and the second pressure sensor module; and automatically calibrating an amount of fluid in the expandable member based on the determined difference in pressure when the determined difference in pressure exceeds the pressure threshold gradient.

Example 49. The method of treatment of any one of the preceding examples, but particularly Example 40, wherein the device is configured to function in an unrestricted blood flow state or a restricted blood flow state, wherein: the unrestricted blood flow state corresponds to the expandable member collapsing to allow blood flow through the blood vessel; and the restricted blood flow state corresponds to the expandable member expanding to reduce blood flow through the blood vessel.

Example 50. A device for modulating blood flow in a blood vessel, the device comprising: an outer frame defining an internal surface; an expandable annulus member at least partially coupled to the internal surface of the outer frame, the expandable annulus member fluidly coupled to a first inflation line, the expandable annulus member defining an annulus volume configured to receive an inflation fluid therein through the first inflation line, wherein the expandable annulus member defines a device orifice through which fluid flows.

Example 51. The device of any one of the preceding examples, but particularly Example 50, wherein the outer frame is a self-expanding frame.

Example 52. The device of any one of the preceding examples, but particularly Example 1, further comprising a first pressure sensor module coupled to a proximal end of the outer frame and configured to detect a first pressure in the blood vessel, with the device adapted to cause, based on the detected first pressure, an increase or a decrease of the inflation fluid in the expandable annulus member.

Example 53. The device of any one of the preceding examples, but particularly Example 52, further comprising a second pressure sensor module coupled to a distal end of the outer frame and configured to detect a second pressure in the blood vessel, with the device adapted to cause, based on a pressure differential between the first pressure and the second pressure, the increase or the decrease of the inflation fluid in the expandable annulus member.

Example 54. The device of any one of the preceding examples, but particularly Example 50, wherein the expandable annulus member comprises a first inner cavity in fluid communication with the first inflation line.

Example 55. The device of any one of the preceding examples, but particularly Example 54, wherein the expandable annulus member comprises a second inner cavity in fluid communication with a second inflation line.

Example 56. The device of any one of the preceding examples, but particularly Example 54, wherein the expandable annulus member comprises a second inner cavity in fluid communication with a second lumen of the first inflation line, wherein a first lumen of the first inflation line is in fluid communication with the first inner cavity.

Example 57. The device of any one of the preceding examples, but particularly Example 1, wherein: the first inflation line is coupled to a subcutaneously implanted reservoir and pump; and actuation of the pump causes the inflation fluid from the reservoir to be pumped into the expandable annulus member to cause partial or full inflation of the expandable annulus member.

Example 58. The device of any one of the preceding examples, but particularly Example 55, wherein: the first inflation line is coupled to a subcutaneously implanted reservoir and pump; the second inflation line is coupled to the subcutaneously implanted reservoir and pump; and actuation of the pump causes the inflation fluid from the reservoir to be pumped into the first cavity and the second cavity of the expandable annulus member to cause partial or full inflation of the expandable annulus member.

Example 59. The device of any one of the preceding examples, but particularly Example 56, wherein: the first lumen of the first inflation line is coupled to a subcutaneously implanted reservoir and pump; the second lumen of the first inflation line is coupled to the subcutaneously implanted reservoir and pump; and actuation of the pump causes the inflation fluid from the reservoir to be pumped into one or both of: the first cavity and the second cavity of the expandable annulus member to cause partial or full inflation of the expandable annulus member.

Example 60. The device of any one of the preceding examples, but particularly Example 58, wherein the first inner cavity is filled prior to the second inner cavity.

Example 61. The device of any one of the preceding examples, but particularly Example 59, wherein the first inner cavity is filled prior to the second inner cavity.

Example 62. The device of any one of the preceding examples, but particularly Example 55, wherein, when filled, the first inner cavity reduces an area of the device orifice to about or above a first predefined threshold.

Example 63. The device of any one of the preceding examples, but particularly Example 62, wherein the first predefined threshold is about 20% to about 60% of the area of the blood vessel in which the device is implanted.

Example 64. The device of any one of the preceding examples, but particularly Example 62, wherein the second inner cavity further reduces the area of the device orifice to about or above a second predefined threshold.

Example 65. The device of any one of the preceding examples, but particularly Example 64, wherein the second predefined threshold is about 5% to about 50% of the area of the blood vessel in which the device is implanted.

Example 66. The device of any one of the preceding examples, but particularly Example 56, the first inner cavity reduces an area of the device orifice to about 20% to about 60% of the area of the blood vessel in which the device is implanted.

Example 67. The device of any one of the preceding examples, but particularly Example 66, wherein the second inner cavity further reduces the area of the device orifice to about 5% to about 50% of the area of the blood vessel in which the device is implanted.

Example 68. The device of any one of the preceding examples, but particularly Example 1, wherein the expandable annulus member is adjustable to a plurality of positions between expanded and contracted, the plurality of positions including at least: an expanded configuration configured to fully occlude the blood vessel; a partially expanded configuration configured to partially occlude the blood vessel; and a contracted configuration configured to not substantially occlude the blood vessel.

Example 69. The device of any one of the preceding examples, but particularly Example 55, wherein the expandable annulus member is adjustable to a plurality of positions between expanded and contracted, the plurality of positions including at least: an expanded configuration configured to fully occlude the blood vessel, wherein the first inner cavity and the second inner cavity are both expanded; a partially expanded configuration configured to partially occlude the blood vessel, wherein the first inner cavity is expanded and the second inner cavity is contracted; and a contracted configuration configured to not substantially occlude the blood vessel, wherein the first inner cavity and the second inner cavity are both contracted.

Example 70. The device of any one of the preceding examples, but particularly Example 56, wherein the expandable annulus member is adjustable to a plurality of positions between expanded and contracted, the plurality of positions including at least: an expanded configuration configured to fully occlude the blood vessel, wherein the first inner cavity and the second inner cavity are both expanded; a partially expanded configuration configured to partially occlude the blood vessel, wherein the first inner cavity is expanded and the second inner cavity is contracted; and a contracted configuration configured to not substantially occlude the blood vessel, wherein the first inner cavity and the second inner cavity are both contracted.

Example 71. A method of treatment for reducing cardiac blood flow at a target site in a blood vessel of a heart of a subject, the method comprising: introducing a device in the blood vessel, the device comprising: an outer frame defining an internal surface; an expandable annulus member at least partially coupled to the internal surface of the outer frame, the expandable annulus member fluidly coupled to one or more inflation lines or an inflation line with one or more lumens, the expandable member defining one or more inner cavities configured to receive an inflation fluid therein through the one or more inflation lines or the inflation line with the one or more lumens; and actuating the device to modulate a flow of blood within the blood vessel.

Example 72. The method of treatment of any one of the preceding examples, but particularly Example 40, further comprising de-actuating the device to maintain or regain the flow of blood through the blood vessel.

Example 73. The method of treatment of any one of the preceding examples, but particularly Example 40, wherein actuating the device causes a partial occlusion of blood flow through the blood vessel.

Example 74. The method of treatment of any one of the preceding examples, but particularly Example 40, further comprising at least one pressure sensor module configured to detect pressure in the blood vessel, with the device adapted to cause a selective increase or a selective decrease of fluid in the expandable annulus member based on the detected pressure.

Example 75. The method of treatment of any one of the preceding examples, but particularly Example 74, wherein the at least one pressure sensor module is coupled to a distal end of the device and configured to measure or sense right atrial pressure.

Example 76. The method of treatment of any one of the preceding examples, but particularly Example 40, wherein the device is configured to function in an unrestricted blood flow state or a restricted blood flow state, wherein: the unrestricted blood flow state corresponds to the expandable annulus member contracting to allow blood flow through the blood vessel; and the restricted blood flow state corresponds to the expandable annulus member expanding to reduce the blood flow through the blood vessel.

Example 77. The method of treatment of any one of the preceding examples, but particularly Example 71, wherein the expandable annulus member includes a first inner cavity and a second inner cavity.

Example 78. The method of treatment of any one of the preceding examples, but particularly Example 77, wherein actuating the device to modulate the flow of blood within the blood vessel includes: expanding the first inner cavity to restrict blood flow through the blood vessel, and selectively expanding the second inner cavity to further restrict the blood flow through the blood vessel.

Example 79. The method of treatment of any one of the preceding examples, but particularly Example 77, wherein actuating the device to modulate the flow of blood within the blood vessel includes: maintaining the first inner cavity in an at least partially expanded configuration to restrict blood flow through the blood vessel, and selectively expanding the second inner cavity to further restrict the blood flow through the blood vessel.

Right atrial deployment may cause desirable pressure changes in other chambers of the heart (e.g., left atrium) or other portions of the patient anatomy. Reductions in pressure in the left atrium are known to help treat symptoms of congestive heart failure. Note that embodiments of the invention may be applicable for deployment in other portions of the anatomy, such as for deployment in and/or modulating flow for the left atrium and/or other portions of the patient anatomy.

As used in the description and claims, the singular form “a”, “an” and “the” include both singular and plural references unless the context clearly dictates otherwise. For example, the term “projection” may include, and is contemplated to include, a plurality of projections. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5 percent, 1 percent or 0.1 percent. All numerical ranges provided herein are inclusive of the stated start and end numbers. The term “substantially” indicates mostly (i.e., greater than 50 percent) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable device for modulating blood flow in a blood vessel, the device comprising:
an expandable member comprising an inflation port coupled to an inflation line, the expandable member defining a volume configured to receive an inflation fluid therein through the inflation port; and
an anchor frame for securing the inflation line along a wall of the blood vessel;
wherein the expandable member restricts flow for modulating blood flow through the blood vessel.

2. The device of claim 1, further comprising at least one pressure sensor module coupled to a distal end of the expandable member and configured to detect pressure in the blood vessel, wherein the device is adapted to cause, based on the detected pressure, an increase or a decrease of the inflation fluid in the expandable member.

3. The device of claim 2, further comprising a safety sensor module configured to interrupt or stop at least one cycle of modulating of blood flow by the device in response to detecting a blood vessel pressure above a predefined safety threshold.

4. The device of claim 1, wherein:
the inflation line is coupled to a subcutaneously implanted reservoir and pump; and
actuation of the pump causes inflation fluid from the subcutaneously implanted reservoir to be pumped into the expandable member to cause partial or full inflation of the expandable member.

5. The device of claim 1, wherein the blood vessel comprises a vena cava, a superior vena cava, or an inferior vena cava.

6. The device of claim 1, wherein the expandable member is adjustable to a plurality of positions between expanded and collapsed, the plurality of positions including at least:
an expanded configuration configured to fully occlude the blood vessel;
a partially expanded configuration configured to partially occlude the blood vessel; and
a collapsed configuration configured to not substantially occlude the blood vessel.

7. The device of claim 1, wherein the expandable member is:
a substantially spherical shape with a substantially annular cross section when in an expanded position; and
configured to inflate up to a maximum elongation of about 900 percent.

8. An implantable flow restrictor for a blood vessel, the implantable flow restrictor comprising:
an outer frame;
an expandable member comprising an inflation port coupled to a first portion of an inflation line, the expandable member defining a volume configured to receive an inflation fluid therein through the inflation port; and
an anchor configured to couple a second portion of the inflation line to an internal surface of the outer frame to allow the expandable member to move within the outer frame or within a portion of the blood vessel in response to blood flow through the blood vessel.

9. The flow restrictor of claim 8, further comprising:
at least one pressure sensor module configured to detect pressure in the blood vessel, wherein the flow restrictor is adapted to cause an increase or a decrease of inflation fluid in the expandable member based on the detected pressure, wherein the at least one pressure sensor module is coupled to a distal end of the inflation line and configured to measure or sense right atrial pressure.

10. The flow restrictor of claim 8, further comprising:
at least one pressure sensor module configured to detect pressure in the blood vessel, wherein the at least one pressure sensor module is coupled to the inflation line, arranged within a threshold distance of a proximal end of the outer frame, and configured to detect superior vena cava pressure or renal pressure;
wherein the flow restrictor is adapted to cause an increase or a decrease of inflation fluid in the expandable member based on the detected pressure.

11. The flow restrictor of claim 8, wherein the expandable member is:
a substantially biconical shape with two substantially congruent cones joined at respective bases; and
configured to inflate up to a maximum elongation of about 300 percent.

12. The flow restrictor of claim 8, wherein the outer frame comprises:
an inflow end, an outflow end, and a lumen defined therethrough,
wherein the lumen comprises a narrowed lumen section that is radially narrowed along an intermediate portion of the outer frame located between the inflow end and the outflow end, the intermediate portion being configured to constrain the expandable member.

13. The flow restrictor of claim 12, wherein the narrowed lumen section enables the expandable member to be selected for a maximum expansion defined by a diameter of the narrowed lumen section.

14. The flow restrictor of claim 8, wherein:
the blood vessel is a superior vena cava; and
the outer frame is configured to be implanted in a vessel branching from the superior vena cava; and
the expandable member is tethered to the outer frame at a length such that the expandable member extends beyond an end perimeter of the outer frame and into the superior vena cava substantially adjacent to an azygous junction.

15. The flow restrictor of claim 14, wherein:
the azygous junction is substantially blocked when the expandable member is in an expanded configuration; and
the azygous junction is substantially unblocked when the expandable member is in a collapsed or deflated configuration.

16. The flow restrictor of claim 8, wherein:
the inflation line is coupled to a subcutaneously implanted reservoir and pump; and
actuation of the pump causes inflation fluid from the reservoir to be pumped into the expandable member to cause partial or full inflation of the expandable member.

17. An implantable device for modulating blood flow in a blood vessel, the device comprising:
an outer frame defining an internal surface;
an expandable annulus member at least partially coupled to the internal surface of the outer frame, the expandable annulus member fluidly coupled to a first inflation line, the expandable annulus member defining an annulus volume configured to receive an inflation fluid therein through the first inflation line, wherein the expandable annulus member defines a device orifice through which fluid flows.

18. The device of claim 17, further comprising:
a first pressure sensor module coupled to a proximal end of the outer frame and configured to detect a first pressure in the blood vessel; and
a second pressure sensor module coupled to a distal end of the outer frame and configured to detect a second pressure in the blood vessel;
wherein the device is adapted to cause, based on a pressure differential between the first pressure and the second pressure, an increase or decrease of the inflation fluid in the expandable annulus member.

19. The device of claim 18, wherein the expandable annulus member comprises:
a first inner annular cavity; and
a second inner annular cavity, wherein the second inner annular cavity is concentrically positioned within the first inner annular cavity;
wherein at least one of the first inner annular cavity and the second inner annular cavity is in fluid communication with the first inflation line, and the first inflation line is coupled to a subcutaneously implanted reservoir and pump.

20. The device of claim 19, wherein:
the first inner annular cavity is in fluid communication with the first inflation line;
the second inner annular cavity is in fluid communication with a second inflation line, and the second inflation line is coupled to the subcutaneously implanted reservoir and pump; and
the pump is adapted to drive the inflation fluid from the reservoir into the first inner annular cavity via the first inflation line and into the second inner annular cavity via the second inflation line to cause partial or full inflation of the expandable annulus member.
